SEQUENCE LISTING (22 sequences)

SEQ ID NO: 1 - DNA, 1545 bp, Homo sapiens
atggccctgagcgagctggcgctggtccgctggctgcaggagagccgccgctcgcggaagctcatcctgttcatcgtgttcctggcgctgctgctggacaacatgctgctcactgtcgtggtccccatcatcccaagttatctgtacagcattaagcatgagaagaatgctacagaaatccagacggccaggccagtgcacactgcctccatctcagacagcttccagagcatcttctcctattatgataactcgactatggtcaccgggaatgctaccagagacctgacacttcatcagaccgccacacagcacatggtgaccaacgcgtccgctgttccttccgactgtcccagtgaagacaaagacctcctgaatgaaaacgtgcaagttggtctgttgtttgcctcgaaagccaccgtccagctcatcaccaaccctttcataggactactgaccaacagaattggctatccaattcccatatttgcgggattctgcatcatgtttgtctcaacaattatgtttgccttctccagcagctatgccttcctgctgattgccaggtcgctgcagggcatcggctcgtcctgctcctctgtggctgggatgggcatgcttgccagtgtctacacagatgatgaagagagaggcaacgtcatgggaatcgccttgggaggcctggccatgggggtcttagtgggcccccccttcgggagtgtgctctatgagtttgtggggaagacggctccgttcctggtgctggccgccctggtactcttggatggagctattcagctctttgtgctccagccgtcccgggtgcagccagagagtcagaaggggacacccctaaccacgctgctgaaggacccgtacatcctcattgctgcaggctccatctgctttgcaaacatgggcatcgccatgctggagccagccctgcccatctggatgatggagaccatgtgttcccgaaagtggcagctgggcgttgccttcttgccagctagtatctcttatctcattggaaccaatatttttgggatacttgcacacaaaatggggaggtggctttgtgctcttctgggaatgataattgttggagtcagcattttatgtattccatttgcaaaaaacatttatggactcatagctccgaactttggagttggttttgcaattggaatggtggattcgtcaatgatgcctatcatgggctacctcgtagacctgcggcacgtgtccgtctatgggagtgtgtacgccattgcggatgtggcattttgtatggggtatgctataggtccttctgctggtggtgctattgcaaaggcaattggatttccatggctcatgacaattattgggataattgatattctttttgcccctctctgcttttttcttcgaagtccacctgccaaagaagaaaaaatggctattctcatggatcacaactgccctattaaaacaaaaatgtacactcagaataatatccagtcatatccgataggtgaagatgaagaatctgaaagtgactga

SEQ ID NO: 2 - DNA, 1545 bp, Homo sapiens
atggccctgagcgagctggcgctggtccgctggctgcaggagagccgccgctcgcggaagctcatcctgttcatcgtgttcctggcgctgctgctggacaacatgctgctcactgtcgtggtccccatcatcccaagttatctgtacagcattaagcatgagaagaatgctacagaaatccagacggccaggccagtgcacactgcctccatctcagacagcttccagagcatcttctcctattatgataactcgactatggtcaccgggaatgctaccagagacctgacacttcatcagaccgccacacagcacatggtgaccaacgcgtccgctgttccttccgactgtcccagtgaagacaaagacctcctgaatgaaaacgtgcaagttggtctgttgtttgcctcgaaagccaccgtccagctcatcaccaaccctttcataggactactgaccaacagaattggctatccaattcccatatttgcgggattctgcatcatgtttgtctcaacaattatgtttgccttctccagcagctatgccttcctgctgattgccaggtcgctgcagggcatcggctcgtcctgctcctctgtggctgggatgggcatgcttgccagtgtctacacagatgatgaagagagaggcaacgtcatgggaatcgccttgggaggcctggccatgggggtcttagtgggcccccccttcgggagtgtgctctatgagtttgtggggaagacggctccgttcctggtgctggccgccctggtactcttggatggagctattcagctctttgtgctccagccgtcccgggtgcagccagagagtcagaaggggacacccctaaccacgctgctgaaggacccgtacatcctcattgctgcaggctccatctgctttgcaaacatgggcatcgccatgctggagccagccctgcccatctggatgatggagaccatgtgttcccgaaagtggcagctgggcgttgccttcttgccagctagtatctcttatctcattggaaccaatatttttgggatacttgcacacaaaatggggaggtggctttgtgctcttctgggaatgataattgttggagtcagcattttatgtattccatttgcaaaaaacatttatggactcatagctctgaactttggagttggttttgcaattggaatggtggattcgtcaatgatgcctatcatgggctacctcgtagacctgcggcacgtgtccgtctatgggagtgtgtacgccattgcggatgtggcattttgtatggggtatgctataggtccttctgctggtggtgctattgcaaaggcaattggatttccatggctcatgacaattattgggataattgatattctttttgcccctctctgcttttttcttcgaagtccacctgccaaagaagaaaaaatggctattctcatggatcacaactgccctattaaaacaaaaatgtacactcagaataatatccagtcatatccgataggtgaagatgaagaatctgaaagtgactga

SEQ ID NO: 3 - protein, 514 aa, Homo sapiens
MALSELALVRWLQESRRSRKLILFIVFLALLLDNMLLTVVVPIIPSYLYSIKHEKNATEIQTARPVHTASISDSFQSIFSYYDNSTMVTGNATRDLTLHQTATQHMVTNASAVPSDCPSEDKDLLNENVQVGLLFASKATVQLITNPFIGLLTNRIGYPIPIFAGFCIMFVSTIMFAFSSSYAFLLIARSLQGIGSSCSSVAGMGMLASVYTDDEERGNVMGIALGGLAMGVLVGPPFGSVLYEFVGKTAPFLVLAALVLLDGAIQLFVLQPSRVQPESQKGTPLTTLLKDPYILIAAGSICFANMGIAMLEPALPIWMMETMCSRKWQLGVAFLPASISYLIGTNIFGILAHKMGRWLCALLGMIIVGVSILCIPFAKNIYGLIAPNFGVGFAIGMVDSSMMPIMGYLVDLRHVSVYGSVYAIADVAFCMGYAIGPSAGGAIAKAIGFPWLMTIIGIIDILFAPLCFFLRSPPAKEEKMAILMDHNCPIKTKMYTQNNIQSYPIGEDEESESD

SEQ ID NO: 4 - protein, 514 aa, Homo sapiens
MALSELALVRWLQESRRSRKLILFIVFLALLLDNMLLTVVVPIIPSYLYSIKHEKNATEIQTARPVHTASISDSFQSIFSYYDNSTMVTGNATRDLTLHQTATQHMVTNASAVPSDCPSEDKDLLNENVQVGLLFASKATVQLITNPFIGLLTNRIGYPIPIFAGFCIMFVSTIMFAFSSSYAFLLIARSLQGIGSSCSSVAGMGMLASVYTDDEERGNVMGIALGGLAMGVLVGPPFGSVLYEFVGKTAPFLVLAALVLLDGAIQLFVLQPSRVQPESQKGTPLTTLLKDPYILIAAGSICFANMGIAMLEPALPIWMMETMCSRKWQLGVAFLPASISYLIGTNIFGILAHKMGRWLCALLGMIIVGVSILCIPFAKNIYGLIALNFGVGFAIGMVDSSMMPIMGYLVDLRHVSVYGSVYAIADVAFCMGYAIGPSAGGAIAKAIGFPWLMTIIGIIDILFAPLCFFLRSPPAKEEKMAILMDHNCPIKTKMYTQNNIQSYPIGEDEESESD

SEQ ID NO: 5 - DNA, 20 bp, Homo sapiens
ccacccttcttcctcctgtt

SEQ ID NO: 6 - DNA, 21 bp, Homo sapiens
cctgcagcctccttctaagat

SEQ ID NO: 7 - DNA, 41 bp, Homo sapiens
catttatggactcatagctccgaactttggagttggttttg

SEQ ID NO: 8 - protein, 30 aa, Homo sapiens
ILCIPFAKNIYGLIAPNFGVGFAIGMVDSS

SEQ ID NO: 9 - protein, 30 aa, Homo sapiens
ILCIPFAKNIYGLIALNFGVGFAIGMVDSS

SEQ ID NO: 10 - protein, 30 aa, Pan troglodytes; identical to SEQ ID NO: 8

SEQ ID NO: 11 - protein, 30 aa, Macaca mulatta; identical to SEQ ID NO: 8

SEQ ID NO: 12 - protein, 30 aa, Mus musculus; identical to SEQ ID NO: 8

SEQ ID NO: 13 - protein, 30 aa, Rattus rattus; identical to SEQ ID NO: 8

SEQ ID NO: 14 - protein, 30 aa, Canis lupus familiaris; identical to SEQ ID NO: 8

SEQ ID NO: 15 - protein, 30 aa, Bos taurus
ILCIPLAKNIYGLIAPNFGVGFAIGMVDSS

SEQ ID NO: 16 - protein, 30 aa, Monodelphis domestica
VLCVPLARNIYGLIAPNFGIGFAIGMVDSS

SEQ ID NO: 17 - protein, 30 aa, Gallus gallus
ILCVPFAKNIYGLIAPNFGVGFAIGMVDSS

SEQ ID NO: 18 - protein, 30 aa, Tetraodon nigroviridis
VICVPFATSIYGLILPNFGVGFAIGMVDSS

SEQ ID NO: 19 - protein, 30 aa, Danio rerio
ILCVPLAKDIYGLIVPNFGVGFAIGMVDSS

SEQ ID NO: 20 - protein, 30 aa, Drosophila melanogaster
LIFIPMATSITHLIIPNAGLGFAIGMVDSS

SEQ ID NO: 21 - protein, 30 aa, Caenorhabditis elegans
LLSIPSATSVAGLIIPHALLGFSIGMIDAS

SEQ ID NO: 22 - protein, 30 aa, Homo sapiens
LLCVPLAMNIFGLIGPNAGLGLAIGMVDSS

(19) United States
(12) Patent Application Publication
(10) Pub. No.: US 2014/0187593 A1
(43) Pub. Date: Jul. 3, 2014

(54) DIAGNOSTIC TEST AND TREATMENT FOR A NEUROLOGICAL DISORDER

(71) Applicant: Berge Minassian, Toronto (CA)

(72) Inventor: Berge Minassian, Toronto (CA)

(21) Appl. No.: 14/096,688

(22) Filed: Dec. 4, 2013

Related U.S. Application Data
(60) Provisional application No. 61/733,210, filed on Dec. 4, 2012.

(51) Int. Cl. C12Q 1/68; G01N 33/68; A61K 31/428

(52) U.S. Cl. CPC C12Q 1/6883; A61K 31/428; G01N 33/6872
USPC 514/367; 506/9; 435/6.12; 435/6.11; 435/6.17; 536/24.33; 536/24.31; 530/350; 435/7.21

(57) ABSTRACT

Methods and compositions relating to diagnosing and treating a VMAT-2 deficiency disease are described. Provided are methods for screening for, diagnosing or detecting a risk of developing a VMAT-2 deficiency disease comprising detecting the presence of a VMAT-2 variant in a sample of a subject, wherein the presence of the VMAT-2 variant is indicative that the subject has a VMAT-2 deficiency disease or an increased risk of developing a VMAT-2 deficiency disease compared to an individual having wild type VMAT-2. Also provided are methods of treating a VMAT-2 deficiency disease with a dopamine agonist.

### RELATED APPLICATIONS

This application claims priority to U.S. Provisional Patent Application No. 61/733,210 filed Dec. 4, 2012, the contents of which are incorporated by reference herein in their entirety.

### FIELD

The disclosure relates to methods and compositions for diagnosing and treating a previously uncharacterized neurological disorder associated with a deficiency in VMAT-2.

### BACKGROUND

Known disorders of biogenic amine neuromediators (dopamine, norepinephrine, epinephrine, and serotonin) comprise defects in nine enzymes¹⁻⁹ and one transporter¹⁰ presenting in early childhood with symptoms referable to the affected neurotransmitter, and diagnosed through measurement of neurotransmitter breakdown products in the cerebrospinal fluid (CSF). Deficiency in dopamine results in movement disorder; deficient norepinephrine and epinephrine cause autonomic dysfunction; and serotonin deficiency leads to sleep and psychiatric disturbance^{2,3,6.}

While most disorders of biogenic amine neuromediators are identified through a CSF neurotransmitter deficiency, members of a family have been identified who present with symptoms of dopamine, serotonin, and/or epinephrine/norepinephrine deficiency but with no demonstrable CSF neurotransmitter deficiency.

One goal of genomic medicine is the tailoring of treatment to the particular pathology of an individual patient. Here, a causative mutation behind an uncharacterized neurological disorder is discovered and the corresponding selection of a treatment based on the underlying biology is described.

### SUMMARY

The inventors describe members of a family with symptoms of dopamine (for example, hypotonia, hypomemia, paucity of movement, oculogyric crises, dystonia), serotonin (sleep and mood disturbance), and epinephrine/norepinephrine (diaphoresis, temperature instability, ptosis, postural hypotension) deficiency, with no demonstrable CSF neurotransmitter deficiency.

Genome investigation revealed a mutation in the gene encoding vesicular monoamine transporter-2 (VMAT-2) that compromises transport of biogenic amines into synaptic vesicles, resulting in deficiency of their synaptic transmission without detectable reductions in their amounts. Surprisingly, treatment of the afflicted subjects with a dopamine agonist resulted in a marked decrease in tremor and dystonic attacks and improvements in other symptoms.

Accordingly, the disclosure relates to a method of screening for, diagnosing and/or detecting an increased risk of developing a VMAT-2 deficiency disease in a subject comprising detecting the presence of a VMAT-2 disease associated variant in a sample of the subject, wherein the presence of a VMAT-2 disease variant is indicative that the subject has a VMAT-2 deficiency disease and/or an increased risk of developing a VMAT-2 deficiency disease. In one embodiment, the method further comprises treating the subject identified as having a VMAT-2 deficiency disease with a dopamine agonist.

Optionally, the VMAT-2 disease associated variant is detected by one or more of: genotyping, using a probe that hybridizes a VMAT-2 disease associated variant, PCR, RT-PCR, NASBA, a binding agent, and/or microarray. In one embodiment, the VMAT-2 disease associated variant is detected by an antibody that selectively binds a VMAT-2 disease associated variant polypeptide, optionally the polypeptide set forth in SEQ ID NO: 4.

The disclosure also relates to a method of screening for, diagnosing and/or detecting an increased risk of developing a VMAT-2 deficiency disease in a human subject comprising:

a) obtaining a sample from the subject;

b) assaying the sample for the presence of and detecting a variant in a VMAT-2 nucleic acid molecule thereby identifying the subject as having a VMAT-2 deficiency disease or an increased risk of developing a VMAT-2 associated disease, the assaying comprising hybridizing a probe and/or primer to the VMAT-2 nucleic acid molecule.

In one embodiment, the VMAT-2 disease associated variant comprises a mutation, optionally a mutation of a nucleotide corresponding to residue 1160 of the VMAT-2 gene.

In another embodiment, the VMAT-2 disease associated variant comprises mutation of a nucleotide corresponding to residue 1160 of SEQ ID No: 1. Optionally, the mutation comprises mutation of a nucleotide to thymidine, for example, cysteine to thymidine.

In a further embodiment, the VMAT-2 disease associated variant comprises a mutation of an amino acid in a VMAT-2 polypeptide. Optionally, the amino acid corresponds to position 387 in a VMAT-2 polypeptide (SEQ ID NO: 3), for example P387L.

In another embodiment, the subject is presymptomatic, has one or more clinical symptoms or clinical features associated with a VMAT-2 deficiency disease and/or has been diagnosed with a VMAT-2 deficiency disease.

In yet another embodiment, the subject has at least one blood relation with a VMAT-2 deficiency disease.

The disclosure also relates to a method of treating a subject with a VMAT-2 deficiency disease comprising administering an effective amount of a dopamine agonist to the subject.

In one embodiment, a method of treating a VMAT-2 deficiency disease is provided comprising:
a) obtaining a sample from the subject;
b) assaying the sample for the presence of and detecting a variant in a VMAT-2 nucleic acid molecule thereby identifying the subject as having a VMAT-2 deficiency disease or an increased risk of developing a VMAT-2 deficiency disease, the assaying comprising hybridizing a probe and/or primer to the VMAT-2 nucleic acid molecule and
c) treating the subject identified as having a VMAT-2 deficiency disease with a dopamine agonist.
In one embodiment, the dopamine agonist is pramipexole.

The disclosure further relates to the use of an effective amount of a dopamine agonist, optionally pramipexole, for treating a subject with a VMAT-2 deficiency disease.

The disclosure also provides an isolated nucleic acid, wherein the nucleic acid hybridizes to:
a. a RNA product of a VMAT-2 variant associated with a VMAT-2 deficiency disease
b. a nucleic acid sequence complementary to a); and/or
c. a nucleic acid sequence corresponding to a).
In one embodiment, the isolated nucleic acid comprises a thymidine at a position corresponding to residue 1160 of the VMAT-2 gene. In another embodiment, the isolated nucleic acid comprises a thymidine at a position corresponding to residue 1160 of SEQ ID NO:1. In one embodiment, the nucleic acid is a cDNA encoding for a VMAT-2 variant associated with a VMAT-2 deficiency disease. In one embodiment, the cDNA encodes a VMAT-2 variant polypeptide with a P387L mutation as set forth in SEQ ID NO: 3.

The disclosure also provides an isolated polypeptide encoded by the isolated nucleic acid described herein. Optionally, the isolated peptide comprises or consists essentially of the amino acid sequence set out in SEQ ID NO:4, including residue 387.

In one embodiment, the isolated nucleic acid is a primer or a probe.

In another embodiment, the isolated nucleic acid comprises or consists of a nucleic acid sequence corresponding to at least 5, 10, 15, 20, 30, 40 or 50 contiguous nucleic acid residues of SEQ ID NO: 2, including residue number 1160, or the complement thereof.

The disclosure also provides a kit for screening for, diagnosing or detecting an increased risk of developing VMAT-2 deficiency disease comprising:
a. a VMAT-2 disease variant detection agent; and
b. instructions for use.

In one embodiment, the detection agent comprises a isolated nucleic acid as described herein. In another embodiment, the detection agent is a probe, optionally comprising all or part of SEQ ID NO: 1, 2, 5 or 6. In a further embodiment, the detection agent is a primer, optionally comprising all or part of any one of SEQ ID NOs: 5 or 6.

In yet another embodiment, the detection agent comprises an antibody. In one embodiment, the antibody is selective for a VMAT-2 variant polypeptide associated with a VMAT-2 deficiency disease. In one embodiment, the antibody is selective for a VMAT-2 variant polypeptide with a P387L mutation as set forth in SEQ ID NO: 3. In one embodiment, there is provided an antibody that is selective for a VMAT-2 variant polypeptide with a P387L mutation as set forth in SEQ ID NO: 3. In one embodiment, the antibody is a monoclonal antibody.

The disclosure also provides a reagent for detecting a VMAT-2 disease associated variant comprising an isolated nucleic acid molecule comprising:
a. any one of SEQ ID NOs: 1, 2, 5 or 6 and/or combinations or parts thereof thereof, and/or
b. a nucleic acid molecule with at least 80%, 90%, 95%, or 99% sequence identity to a);

wherein the nucleic acid molecule is capable of binding a VMAT-2 disease associated variant under stringent hybridization conditions.

The disclosure also relates to the use of a dopamine agonist therapy for a subject comprising a VMAT-2 disease associated variant, wherein the presence of the VMAT-2 disease associated variant, optionally P387L or 1160C>T, is detected according to any one of methods described herein.

Other features and advantages of the disclosure will become apparent from the following detailed description. It should be understood, however, that the description and the specific examples while indicating preferred embodiments are given by way of illustration only, since various changes and modifications within the spirit and scope of the disclosure will become apparent to those skilled in the art from this description of various embodiments.

### DRAWINGS

Embodiments are described below in relation to the drawings in which:

FIG. 1 depicts the causative defect in VMAT2. Panel A depicts the family pedigree. Panel B shows the homozygous haplotype shared by the affected individuals. Panel C shows electropherograms of a portion of the SLC18A2 gene containing the mutation. Panel D depicts the VMAT2 protein within the synaptic vesicle membrane (adapted from Erikson and Eiden, J Neurochem 61, 214-2317 (1993)); the arrow indicates the mutated proline. Panel E shows the primary sequence of the portion of human VMAT2 which in our patients contains the mutated proline, and corresponding sequences in various orthologs and in the VMAT1 paralog of neuroendocrine cells. TM9 and TM10 are parts of the ninth and tenth transmembrane domains of VMAT2 respectively.

FIG. 2 shows monoamine metabolism, vesicular loading, and synaptic transmission, and loading defect in the subjects. Panel A summarizes the metabolism of monoamines and their transport into synaptic vesicles; of note that norepinephrine and epinephrine are synthesized from dopamine within the vesicle; SR, sepiapterin reductase; BH4, tetrahydrobiopterin; qBH2, quinonoid dihydrobiopterin; OMD, O-methldopa; AADC, aromatic amino acid decarboxylase; MAO, monoamine oxidase; COMT, catechol-O-methyl transferase; HVA, homovanilic acid; HIAA, hydroxyindoleacetic acid; VMAT2, vesicular monoamine transporter-2; Dr3H, dopamine beta-hydroxylase; NE, norepinephrine; MHPG, 3-methoxy-4-hydroxyphenylglycol; PNMT, phenylethanolamine N-methyltransferase; VMA, vanillylmandelic acid. Panel B depicts monoamine neurotransmission; the site of action of the dopamine agonist used to treat the patients described herein is at the postsynaptic neuron membrane. Panel C is a time course of tritiated serotonin uptake into vesicles prepared from Cos cells transfected with wild-type (wt) or mutant (P387L) human VMAT2, or vector alone (control). Western blot demonstrates equivalent VMAT2 levels and posttranslational processing in assay lysates. Panel D shows uptake of tritiated serotonin (10 min) by vesicles prepared from cells transfected with wt and P387L-VMAT2, with and without the addition of 10 μM of the specific VMAT inhibitor reserpine.

FIG. 3 shows the nucleic acid and polypeptide sequences for wild type and variant VMAT-2 as well as exemplary forward and reverse primers useful for the detection of a VMAT-2 disease associated variant.

### DETAILED DESCRIPTION

The present inventors have discovered that a variant in vesicular monoamine transporter 2 (VMAT-2) is associated with a previously uncharacterized neurological disorder. Based on an understanding of the molecular basis behind the disorder, it was unexpectedly discovered that the disorder can be treated with dopamine agonists.

Accordingly, an aspect of the disclosure provides a method of screening for, diagnosing and/or detecting a VMAT-2 deficiency disease or an increased risk of developing a VMAT-2 deficiency disease in a subject comprising detecting the presence of a VMAT-2 disease associated variant in a sample of the subject, wherein the presence of the VMAT-2 disease associated variant is indicative of the disorder described herein and/or an increased risk of developing the disorder.

In one aspect of the disclosure, the method comprises detecting the homozygous presence of a VMAT-2 disease associated variant in a sample of the subject, wherein the homozygous presence of the VMAT-2 disease associated variant is indicative of the disorder described herein and/or an increased risk of developing the disorder.

In another aspect of the disclosure, the method comprises detecting the heterozygous presence of a VMAT-2 disease associated variant in a sample of the subject, wherein the heterozygous presence of the VMAT-2 disease associated variant is indicative of the disorder described herein and/or an increased risk of developing the disorder. As used herein, “VMAT-2 deficiency disease” refers to a disease wherein the disease is associated and/or caused by deficiencies in the function of VMAT-2. In one embodiment, VMAT-2 deficiency disease is associated with and/or caused by mutations in the locus encoding VMAT-2. One example of a “VMAT-2 associated disease” is a movement disorder, optionally an infantile-onset movement disorder including severe tremor, rigidity, and non-ambulation, mood disturbance, autonomic instability, and developmental delay. In another example, a VMAT-2 deficiency disease is a disease wherein the subject has symptoms of dopamine, serotonin and epinephrine/nor epinephrine deficiency but there is no demonstrable or observable CSF neurotransmitter deficiency. In one embodiment, a VMAT-2 associated disease is associated with and/or caused by homozygous mutations at the VMAT-2 loci. The term “VMAT-2 associated disease” also encompasses diseases associated with heterozygous mutations at the VMAT-2 loci, for example diseases with milder symptoms and/or fewer symptoms than those seen in diseases associated with the homozygous state. For example, in one embodiment, depression is a VMAT-2 deficiency disease. In another embodiment, a VMAT-2 deficiency disease is a disease that responds to dopamine agonists but is worsened with dopamine antagonists.

As used herein, “homozygous” refers to having two of the same alleles at a particular genetic locus, for example, two alleles of a VMAT-2 disease variant at the VMAT-2 gene loci. As used herein, “heterozygous” refers to having two different alleles at a particular genetic locus, for example, one allele of wild-type VMAT-2 and one allele of a VMAT-2 disease associated variant at the VMAT-2 gene loci.

As used herein the phrase “screening for, diagnosing or detecting a VMAT-2 deficiency disease” refers to a method or process of determining if a subject has a VMAT-2 deficiency disease.

As used herein the phrase “screening for, diagnosing or detecting a risk of developing a VMAT-2 deficiency disease” refers to a method or process of determining if a subject has an increased risk of developing a VMAT-2 deficiency disease.

As used herein “VMAT-2” or “vesicular monoamine transporter 2” refers to a VMAT-2 gene including gene introns and its gene products including transcribed nucleic acids and translated polypeptides (such as VMAT-2 gene, VMAT-2 transcripts, VMAT-2 polypeptides). VMAT-2 is also known as SLC18A2. VMAT-2 optionally refers to the full sequence or a portion thereof which retains VMAT-2 activity. In one embodiment VMAT-2 refers to human VMAT-2.

“Wild-type VMAT-2 gene” or “wild-type VMAT-2 gene products” as used herein refers to common naturally occurring forms of the VMAT-2 gene or gene products that are not associated with disease. In one embodiment, wild type VMAT-2 has the sequence, or part of the sequence, provided at SEQ ID NO. 1, wherein the nucleotide at residue 1160 is cytosine. In one embodiment, wild type VMAT-2 has the mRNA sequence identified by Genbank Accession number NM_{—}003054. In another embodiment wild type VMAT-2 has the protein sequence identified by Genbank Accession number

NP_{—}003045 protein. In another embodiment, wildtype VMAT-2 has the amino acid sequence provided at SEQ ID NO: 3, wherein the amino acid at reside

387 is proline. Protein and polypeptide are used herein interchangeably. Various isoforms of VMAT-2 exist.

As used herein “VMAT-2 disease associated variant” or “VMAT-2 disease variant” means any VMAT-2 molecule, nucleic acid, including an allele, or polypeptide that comprises at least one modification and/or alteration compared to wild-type VMAT-2 that is associated with or useful for screening, diagnosing or detecting an increased risk of developing a VMAT-2 deficiency disease. The modification and/or alteration is optionally a VMAT-2 gene mutation, for example a germline mutation. As used herein, a “VMAT-2 gene mutation” refers to a nucleotide change (and/or nucleotide changes) in the VMAT-2 gene allele or alleles that is/are reflected in nucleic acid and polypeptide gene products that is/are not present in wild-type VMAT-2 gene or gene products which are not associated with disease. The gene mutation is in one embodiment inherited e.g. a germline mutation. In another embodiment, the gene mutation is sporadic (eg. a somatic mutation). VMAT-2 gene mutations include without limitation, missense mutations, deletion mutations, point mutations, and/or insertion mutations. Accordingly VMAT-2 gene mutations include nucleotide polymorphisms such as single nucleotide polymorphisms associated with disease.

In an embodiment, the VMAT-2 disease associated variants comprise or consist of VMAT-2 polypeptide mutated at proline 387 and/or VMAT-2 mRNA mutated at cytosine 1160, and/or VMAT-2 according to SEQ ID NO:1 mutated at cytosine 1160, including missense mutations, deletions, and insertions.

In an embodiment, the VMAT-2 disease associated variants comprise P387L or 1160C>T. In another embodiment, the VMAT-2 associated variant is a change from C to T at residue 1160 of SEQ ID NO. 1.

In another embodiment, the VMAT-2 disease associated variant is a change from C to T at the residue indicated in bold and underlined in the sequence below:

CATTTATGGACTCATAGCTCCGAACTTTGGAGTTGGTTTTG

In another embodiment, the VMAT-2 disease associated variant is selected from P387L or 1160C>T. In another embodiment, the VMAT-2 disease associated variant comprises a P387L mutation. In a further embodiment, the VMAT-2 disease associated variant comprises a 1160C>T mutation.

In another embodiment, the VMAT-2 disease associated variant is a variant that is within, or adjacent to, a transmembrane segment of the VMAT-2 polypeptide. In a further embodiment, the VMAT-2 disease associated variant is a variant that results in a VMAT-2 polypeptide that exhibits decreased protein processing, for example decreased monoamine transport, compared to a wild-type VMAT-2 polypeptide.

A person skilled in the art would recognize that the genomic mutation corresponding to the mutations described herein is optionally detected in the opposite DNA strand. A person skilled in the art will understand that primers, probes and other reagents can be designed to detect the corresponding mutation in the non-coding allele.

VMAT-2 gene mutations are readily detected by analyzing the VMAT-2 gene or its gene products. For example nucleic acids and/or polypeptides corresponding to a VMAT-2 gene are optionally sequenced and compared to corresponding wild-type sequences. Gene mutations are optionally detected by analyzing genomic sequence.

The term “corresponding to” as used herein means situated in a different sequence position but having sequence characteristics in common, including identical, or substantially identical, nucleotide sequence flanking the mutation (eg. substantial identity is optionally at least 75% identity over four or more contiguous nucleotides). For example, “a nucleotide corresponding to position 1160 in VMAT-2 mRNA” or “a nucleotide corresponding to nucleotide 1160 in SEQ ID NO: 1” refers to a nucleotide that is equivalently situated in terms of flanking sequence and relative position in VMAT-2 but that may be identified by a different nucleotide number in a different transcript. Further “corresponding to” can refer to derived from or related to, for example a nucleic acid corresponding to a gene refers to a nucleic acid derived from the gene such as a transcript and/or an amplified or synthetic copy related to the gene. Similarly, an amino acid sequence corresponding to a nucleic acid refers to an amino acid that is coded for by the nucleic acid.

Gene mutations are optionally detected by analyzing nucleic acids corresponding to the VMAT-2 gene such as RNA transcripts e.g. mRNA or complementary DNA (cDNA). In the general population, the predominant nucleotide found in VMAT-2 mRNA at position 1160 in subjects without a VMAT-2 associated disease or an increased risk of developing a VMAT-2 deficiency disease is cytosine. The inventors have shown that the nucleotide found at position 1160 in VMAT-2 mRNA is modified in subjects with a VMAT-2 deficiency disease or an increased risk of developing a VMAT-2 deficiency disease, from cytosine (C) to thymidine (T). The inventors have shown that this mutation is a missense mutation. The VMAT-2 disease variant comprising this gene mutation is optionally referred to as 1160C>T. Accordingly, in one embodiment the VMAT-2 disease variant detected comprises a gene mutation in a nucleotide corresponding to position 1160 in VMAT-2 mRNA. Accordingly, in another embodiment the VMAT-2 disease variant detected comprises a gene mutation in a nucleotide corresponding to position 1160 in SEQ ID NO:1. In another embodiment, the VMAT-2 disease variant comprises or consists of SEQ ID:2, or a part thereof, including residue 1160. In one embodiment, the gene mutation is a missense mutation. In another embodiment, the nucleotide detected at position 1160 is thymidine. In yet a further embodiment, the VMAT-2 variant is 1160C>T. A person skilled in the art will recognize nucleotide mutations in mRNA can be detected using corresponding cDNA. Further a person skilled in the art will recognize that mutations at this position such as deletion of one or more nucleotides comprising the nucleotide at position 1160 will also be associated with a VMAT-2 deficiency disease or an increased risk of developing a VMAT-2 deficiency disease. Similarly, it is expected that modification of this nucleotide to guanine or adenosine would also be associated with a VMAT-2 deficiency disease or an increased risk of developing a VMAT-2 deficiency disease. Accordingly in one embodiment, the modification comprises a deletion of the nucleotide at position 1160 in a VMAT-2 mRNA. In another embodiment, the nucleotide detected at 1160 is guanine or adenine.

Gene mutations are optionally detected by analyzing polypeptides corresponding to the VMAT-2 gene. The inventors have shown that the amino acid found at position 387 in VMAT-2 protein (SEQ ID NO:3) is modified in subjects with a VMAT-2 associated disease or an increased risk of developing a VMAT-2 associated disease from proline (Pro) to leucine (Leu). The VMAT-2 disease associated variant comprising this mutation is optionally referred to as Pro387Leu and or P387L. Accordingly, in an embodiment the VMAT-2 disease associated variant detected comprises a modification in the amino acid corresponding to position 387 in VMAT-2 polypeptide. In another embodiment, the amino acid detected at position 387 is leucine (SEQ ID NO:4). In yet a further embodiment, the VMAT-2 disease associated variant is P387L. A person skilled in the art will recognize that modifications at this position such as deletion of one or more amino acids comprising the amino acid at position 387 will also be associated with VMAT-2 deficiency disease or an increased risk of developing VMAT-2 deficiency disease. Similarly, it is expected that modification of this amino acid to other branched amino acids would also be associated with VMAT-2 deficiency disease or an increased risk of developing VMAT-2 deficiency disease. For example, it is shown that a nucleotide change in VMAT-2 mRNA at position 1160, results in introduction of leucine (i.e nucleotide change to thymidine) as mentioned above. A person skilled in the art will understand that additional amino acid changes result from changes in the first and third nucleotides of the codon coding for the amino acid at position 387. Accordingly in one embodiment, the modification comprises a deletion of amino acid at position 387 in VMAT-2 polypeptide. In another embodiment the amino acid at 387 is replaced by a stop codon.

Other modifications can include post-translational modifications of proline 387 in VMAT-2.

A person skilled in the art will understand that positions of mutations provided are relative to the particular accession numbers and SEQ ID NOS provided. A person skilled in the art would readily be able to determine corresponding position in any VMAT-2 isoforms, VMAT-2 homologues, VMAT-2 sequence fragments or other related sequences.

The terms “risk” and “increased risk” as used herein refer to a subject having a predisposition to developing a disease e.g increased risk compared to the average risk of a population. The predisposition is optionally inherited, or optionally acquired (e.g sporadic mutation). The increased risk is relative to a subject not having a VMAT-2 disease associated variant.

The term “sample” and “sample of a subject” as used herein refer to any sample of a subject that comprises nucleic acids or polypeptide and/or includes sequence or sequence data corresponding to VMAT-2 gene, RNA or protein sequence. For example, a priori sequenced VMAT-2 gene, RNA or protein sequence is optionally used to detect VMAT-2 disease associated variants. In one embodiment, the sample comprises blood, whole blood or a fraction thereof. In another embodiment, the sample is selected from the group consisting of fresh tissue such as a biopsy, frozen tissue and paraffin embedded tissue. In other embodiments, the sample comprises any nucleated cell from the human body and any cell lines generated to express VMAT-2.

The term “subject” as used herein includes all members of the animal kingdom including multicellular organisms, including mammals, and preferably means humans.

VMAT-2 deficiency diseases are difficult to diagnose. The inventors have determined that the methods described herein identify individuals presymptomatically. Accordingly, in one embodiment, the individual is presymptomatic.

As used herein, “a relative” or “blood relation” is a relative genetically related, or related by birth, and includes without limitation 1^{st}, 2^{nd}, 3^{rd}, 4^{th}, 5^{th}, 6^{th}, 7^{th},8^{th}, 9^{th} and 10^{th} degree relations, for example but not limited to parents, children, grandchildren, grandparents, cousins and/or 2^{nd} cousins related by blood.

### Isolated Nucleic Acids and Compositions

VMAT-2 disease associated variants are readily detected using isolated nucleic acids and/or compositions comprising isolated nucleic acids or polypeptides that are specific for a VMAT-2 disease associated variant.

Accordingly in one aspect, the application provides isolated nucleic acids useful for detecting VMAT-2 disease associated variants and compositions comprising isolated nucleic acids useful for detecting VMAT-2 disease associated variants. Another aspect provides an isolated nucleic acid molecule comprising a nucleic acid sequence comprising a VMAT-2 gene or transcript disease associated variant.

The term “isolated nucleic acid sequence” and/or “oligonucleotide” as used herein refers to a nucleic acid substantially free of cellular material or culture medium when produced by recombinant DNA techniques, or chemical precursors, or other chemicals when chemically synthesized. The term “nucleic acid” and/or “oligonucleotide” as used herein refers to a sequence of nucleotide or nucleoside monomers consisting of naturally occurring bases, sugars, and intersugar (backbone) linkages, and is intended to include DNA and RNA which can be either double stranded or single stranded, represent the sense or antisense strand. The term also includes modified or substituted oligomers comprising non-naturally occurring monomers or portions thereof, which function similarly, which are referred to herein as “chemical analogues” and/or “oligonucleotide analogues” such as “peptide nucleic acids”. Such modified or substituted nucleic acids may be preferred over naturally occurring forms because of properties such increased stability in the presence of nucleases.

One aspect of the application provides an isolated nucleic acid molecule, wherein the isolated nucleic acid molecule hybridizes to:
a. a RNA product of a VMAT-2 variant associated with a VMAT-2 deficiency disease
b. a nucleic acid sequence complementary to a); and/or
c. a nucleic acid sequence corresponding to a).

Optionally, the isolated nucleic acid molecule is used to detect a VMAT-2 disease associated variant.

In an embodiment, a) comprises a VMAT-2 disease associated variant. In an embodiment, the nucleic acid complementary to or corresponding to a) comprises genomic sequence.

The term “hybridize” refers to the sequence specific non-covalent binding interaction with a complementary nucleic acid. One aspect of the application provides an isolated nucleotide sequence, which hybridizes to a RNA product of VMAT-2 or a nucleic acid sequence which is complementary to an RNA product of a gene of VMAT-2. In one embodiment the hybridization is conducted under at least moderately stringent conditions. In a preferred embodiment, the hybridization is under high stringency conditions. Appropriate stringency conditions which promote hybridization are known to those skilled in the art, or can be found in Current Protocols in Molecular Biology, John Wiley & Sons, N.Y. (1989), 6.3.1 6.3.6. For example, 6.0× sodium chloride/sodium citrate (SSC) at about 45° C. for 15 minutes, followed by a wash of 2.0×SSC at 50° C. for 15 minutes may be employed.

The stringency may be selected based on the conditions used in the wash step. For example, the salt concentration in the wash step can be selected from a high stringency of about 0.2×SSC at 50° C. for 15 minutes. In addition, the temperature in the wash step can be at high stringency conditions, at about 65° C. for 15 minutes.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.−16.6 (Log 10 [Na+])+0.41(% (G+C)−600/I), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% sequence identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at Tm−5° C. based on the above equation, followed by a wash of 0.2×SSC/0.1% SDS at 60° C. for 15 minutes. Moderately stringent hybridization conditions include a washing step in 3×SSC at 42° C. for 15 minutes. It is understood, however, that equivalent stringencies may be achieved using alternative buffers, salts and temperatures. Additional guidance regarding hybridization conditions may be found in: Current Protocols in Molecular Biology, John Wiley & Sons, N.Y., 1989, 6.3.1-6.3.6 and in: Sambrook et al., Molecular Cloning, a Laboratory Manual, Cold Spring Harbor Laboratory Press, 2000, Third Edition.

The term “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present application. BLAST protein searches can be performed with the XBLAST program parameters set, e.g., to score-50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). The percent identity between two sequences can be determined using techniques similar to those described above, with or without allowing gaps. In calculating percent identity, typically only exact matches are counted. In an embodiment, the isolated nucleic acids are useful as primers.

The term “primer” as used herein refers to a nucleic acid sequence, whether occurring naturally as in a purified restriction digest or produced synthetically, which is capable of acting as a point of synthesis when placed under conditions in which synthesis of a primer extension product, which is complementary to a nucleic acid strand is induced (e.g. in the presence of nucleotides and an inducing agent such as DNA polymerase and at a suitable temperature and pH). The primer must be sufficiently long to prime the synthesis of the desired extension product in the presence of the inducing agent. The exact length of the primer will depend upon factors, including temperature, sequences of the primer and the methods used. A primer typically contains 15-25 or more nucleotides, although it can contain less, for example, up to 5, 10, 12 or 15 nucloetides. The factors involved in determining the appropriate length of primer are readily known to one of ordinary skill in the art.

Accordingly, one aspect of the disclosure provides a reagent for detecting and/or amplifying a VMAT-2 disease associated variant, such as an isolated nucleic acid primer. In an embodiment, the isolated nucleic acid molecule comprises:

a) any one of SEQ ID NOs: 1, 2, 5 and 6 or the complement thereof, and/or combinations or parts thereof; and/or

b) a nucleic acid molecule with at least 80%, 90%, 95%, or 99% sequence identity to a), characterized in that the nucleic acid molecule is capable of binding VMAT-2 under moderately stringent conditions. In an embodiment, the nucleic acid with at least 80%, 90%, 95%, or 99% sequence identity to a) binds VMAT-2 under moderately stringent conditions and is capable of priming strand synthesis. Isolated nucleic acid molecules including for example SEQ ID NOs: 5 and 6 are useful as primers to amplify VMAT-2. In an embodiment, the isolated nucleic acid molecule is an amplified nucleic acid which is produced by amplification of a VMAT-2 disease associated variant containing template. Optionally, the isolated nucleic acids are at least 5, 10, 15, 20, 30, 40, 50, 60, 70, 80, 90 or 100 nucleotides long. In another embodiment, they are 5-80, 10-60 or 15-30 nucleotides long.

The inventors designed a number of primers useful for detecting VMAT-2 disease variants as provided at SEQ ID NOS: 5 and 6

In another aspect, the application describes probes that are useful for detecting a VMAT-2 disease associated variant. Where the variation is only a single nucleotide change, for example 1160C>T, shorter probes used at high stringency are useful. For example, oligonucleotide probes having a sequence length ranging from 16 to 20 nucleotides, comprising, within the sequence, for example, at the centre, a nucleotide specific for the allelic variants of the gene coding for a VMAT-2 disease associated variant, wherein the oligonucleotide probes hybridizes with the VMAT-2 disease associated variant.

The term “probe” as used herein refers to a nucleic acid sequence that will hybridize to a nucleic acid target sequence. In one example, the probe hybridizes to an RNA VMAT-2 disease associated variant or a nucleic acid sequence complementary to the RNA VMAT-2 disease associated variant. The length of probe depends on the hybridize conditions and the sequences of the probe and nucleic acid target sequence. In one embodiment, the probe is at least 8, 10, 15, 20, 25, 50, 75, 100, 150, 200, 250, 400, 500 or more nucleotides in length.

Accordingly, one aspect of the disclosure provides a reagent for detecting a VMAT-2 disease variant comprising an isolated nucleic acid molecule comprising:

a) any one of SEQ ID NOs: 1, 2, 5 and 6 or the complement thereof, and/or combinations or parts thereof; and/or

b) a nucleic acid molecule with at least 80%, 90%, 95%, or 99% sequence identity to a), characterized in that the nucleic acid molecule is capable of binding a VMAT-2 disease variant under stringent conditions. In a further embodiment, the nucleic acid molecule does not bind wild type VMAT-2 under stringent hybridization conditions.

A person skilled in the art will recognize that all or part of the above probes can be used.

In certain embodiments the isolated nucleic acid comprises a detectable label, such as a fluorescent or radioactive label.

### Detection Methods for the Presence of a VMAT-2 Mutation

The presence of a VMAT-2 disease associated variant are readily detected using methods that detect a gene mutation in a VMAT-2 disease associated variant gene, RNA gene product, such as VMAT-2 transcripts, and/or polypeptide gene product.

### Detecting Nucleic Acid VMAT-2 Disease Associated Variants

A person skilled in the art will appreciate that a number of methods are useful for detect the presence of a VMAT-2 disease associated variant in a VMAT-2 nucleic acid.

For example a variety of techniques are known in the art for detecting a gene mutation or alteration within a sample, including genotyping, microarrays, Restriction Fragment Length Polymorphism, Southern Blots, SSCP, dHPLC, single nucleotide primer extension, allele-specific hybridization, allele-specific primer extension, oligonucleotide ligation assay, and invasive signal amplification, Matrix-assisted laser desorption/ionization time-of-flight (MALDI-TOF) mass spectrometry, and Fluorescence polarization (FP). Such methods optionally employ the isolated nucleic acid compositions of the disclosure. The VMAT-2 disease associated variants, such as germline alterations, are readily detected by VMAT-2 gene analysis. For example, this can be accomplished by gene amplification analysis such as polymerase chain reaction (PCR) analysis of VMAT-2 or a part thereof, optionally followed by sequencing, and comparing the VMAT-2 amplification profile or sequence to a wild-type VMAT-2 amplification profile or wild-type VMAT-2 sequence. In an embodiment, one or more VMAT-2 exons are amplified by PCR, and analyzed for gene mutations, for example by single-strand conformation polymorphism (SSCP). In an embodiment, one or more VMAT-2 exons are sequenced and analyzed for gene mutations, for example by comparing the sequence obtained from a sample of a subject, to wild-type VMAT-2 sequence. The full exon or a part thereof, for example a part known to be associated with disease, for example a part comprising a nucleotide corresponding to the nucleotides described herein is optionally PCR amplified and/or sequenced. Detecting “T” at position 1160 of VMAT-2 or at position 1160 of SEQ ID NO: 1 is indicative of having a VMAT-2 deficiency disease or an increased risk of developing a VMAT-2 deficiency disease. In another embodiment, 2, 3, 4, 5, 6, 7, 8, 9, 10, 11, or 12 exons, or parts thereof, are PCR amplified and/or sequenced and the PCR profile and/or sequence analyzed for gene mutations. Accordingly, the presence of a VMAT-2 gene mutation is detected in one embodiment by sequencing. Methods of sequencing are well known in the art. In one method, primers flanking a VMAT-2 mutation are selected, for example primers which amplify exon 13 either in genomic sequence and/or corresponding transcripts, and used to amplify the gene region comprising a VMAT-2 mutation. The amplified region is sequenced and analysed. Gene mutations can be detected by detecting the presence of the mutation in the gene or in a corresponding transcribed sequence. A variety of techniques are known in the art that are suitable for detecting mutations in the gene or in a corresponding transcribed sequence.

For example, primers which span one or more exons that comprise the putative location of a VMAT-2 disease associated variant, are useful to detect VMAT-2 disease associated variants. In an embodiment, a composition comprising SEQ ID NO: 5 and SEQ ID NO: 6 is used to detect mutations at position 1160 of VMAT-2 and/or position 1160 of SEQ ID NO:1, such as 1160C>T. A person skilled in the art readily designs and uses suitable additional primers based on the sequences provided herein.

In another embodiment, VMAT-2 disease associated variants are readily detected by polymerase chain reaction (PCR), real time PCR, multiplex ligation dependent probe amplification (MLPA), nucleic acid sequence based amplification (NASBA) and/or real time NASBA. As used herein “NASBA” refers to a sensitive isothermal transcription-based amplification method used for example for RNA research. NASBA technology is optionally applied to single nucleotide polymorphism (SNP) analysis using human genomic DNA as a template. For example combination of DNA NASBA with multiplex hybridization of specific molecular beacons makes it possible to discriminate the presence of mutations of interest (Berard, C, Cazalis M A, Leissner P, Mougin B., DNA nucleic acid sequence-based amplification-based genotyping for polymorphism analysis. Biotechniques. 2004, 37:680-2, 684, 686).

In another embodiment, the method of detecting the presence of a VMAT-2 disease variant comprises a probe that specifically hybridizes a VMAT-2 disease associated variant. The probe optionally hybridizes to an mRNA sequence, corresponding complementary DNA or copy DNA (cDNA) or a genomic sequence. The probe can hybridize a VMAT-2 mutation directly or an amplified product comprising the VMAT-2 gene mutation. In another embodiment the probe binds upstream or downstream of a VMAT-2 gene mutation. For example, in one embodiment an amplified region comprising a VMAT-2 gene mutation is hybridized using a composition comprising a probe specific for the VMAT-2 gene mutation (e.g. “T”) under stringent hybridization conditions. In an embodiment the probe comprises all or part (e.g. 10-19 nucleotides, or any number in between) of SEQ ID NO:1, 2, 5 or 6 to detect the disease variation. A person skilled in the art would recognize that a probe that hybridizes to a sequence in the non-coding (and/or corresponding sequence such as cDNA) comprising the VMAT-2 disease associated variant is also useful with the methods, compositions and kits described herein.

In one embodiment, PCR or RT-PCR is employed to detect the presence of a VMAT-2 mutation. For example, PCR and RT-PCR and primers flanking the mutation are employed to amplify VMAT-2 gene sequence and transcript sequence respectively in a sample comprising DNA (for PCR) or RNA (for RT-PCR). The amplified products are optionally sequenced to determine if a VMAT-2 disease associated variant is present in the sample.

In another embodiment, the method of detecting the presence of a VMAT-2 mutation comprises use of a restriction enzyme. For example amplified products can be digested with a restriction enzyme that specifically recognizes sequence comprising a VMAT-2 disease associated variant but does not recognize sequence corresponding to the wild-type or non-disease associated VMAT-2.

### Detecting Polypeptide VMAT-2 Disease Associated Variants

A person skilled in the art will recognize that there are several methods known in the art for detecting a polypeptide VMAT-2 disease associated variant.

A polypeptide VMAT-2 disease associated variant is optionally detected using a binding agent that specifically binds a VMAT-2 disease associated variant polypeptide gene products and not wild type VMAT-2 polypeptide gene products. In one embodiment, the binding agent is an isolated polypeptide.

The term “isolated polypeptide” as used herein refers to a proteinaceous agent, such as a peptide, polypeptide or protein, which is substantially free of cellular material or culture medium when produced recombinantly, or chemical precursors, or other chemicals, when chemically synthesized.

The phrase “bind to polypeptide products” as used herein refers to binding agents such as isolated polypeptides that specifically bind to VMAT-2 disease associated variants described in the application. In an embodiment, isolated polypeptides are antibodies or antibody fragments.

The term “antibody” as used herein is intended to include monoclonal antibodies, polyclonal antibodies, and chimeric antibodies. The antibody may be from recombinant sources and/or produced in transgenic animals. The term “antibody fragment” as used herein is intended to include Fab, Fab′, F(ab′)₂, scFv, dsFv, ds-scFv, dimers, minibodies, diabodies, and multimers thereof and bispecific antibody fragments. Antibodies can be fragmented using conventional techniques. For example, F(ab′)₂ fragments can be generated by treating the antibody with pepsin.

To produce human monoclonal antibodies, antibody producing cells (lymphocytes) can be harvested from a human having cancer and fused with myeloma cells by standard somatic cell fusion procedures thus immortalizing these cells and yielding hybridoma cells. Such techniques are well known in the art, (e.g. the hybridoma technique originally developed by Kohler and Milstein (Nature 256:495-497 (1975)) as well as other techniques such as the human B-cell hybridoma technique (Kozbor et al., Immunol.Today 4:72 (1983)), the EBV-hybridoma technique to produce human monoclonal antibodies (Cole et al., Methods Enzymol, 121:140-67 (1986)), and screening of combinatorial antibody libraries (Huse et al., Science 246:1275 (1989)). Hybridoma cells can be screened immunochemically for production of antibodies specifically reactive with VMAT-2 disease associated variants and the monoclonal antibodies can be isolated.

Specific antibodies, or antibody fragments, reactive against particular VMAT-2 disease associated antigens, may also be generated by screening expression libraries encoding immunoglobulin genes, or portions thereof, expressed in bacteria with cell surface components. For example, complete Fab fragments, VH regions and FV regions can be expressed in bacteria using phage expression libraries (See for example Ward et al., Nature 341:544-546 (1989); Huse et al., Science 246:1275-1281 (1989); and McCafferty et al., Nature 348:552-554 (1990)).

In one embodiment isolated polypeptides, antibodies or antibody fragments are used to detect a VMAT-2 disease associated variant. In one embodiment the isolated polypeptides, antibodies or antibody fragments are labeled with a detectable marker.

The label is preferably capable of producing, either directly or indirectly, a detectable signal. For example, the label may be radio-opaque or a radioisotope, such as ³H, ¹⁴C, ³²P ³⁵S ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion.

In another embodiment, the detectable signal is detectable indirectly. For example, a secondary antibody that is specific for the isolated protein described in the application and contains a detectable label is useful to detect the isolated polypeptide described in the application.

A person skilled in the art will appreciate that a number of methods can be used to detect a polypeptide VMAT-2 disease associated variant, including immunoassays such as Western blots, ELISA, and immunoprecipitation followed by SDS-PAGE, as well as immunocytochemistry or immunohistochemistry.

In an embodiment the binding agents are fixed to a solid support. In a further embodiment the solid support is an ELISA plate.

In one embodiment, there is provided an antibody that selectively binds to a VMAT-2 variant polypeptide as described herein. In one embodiment, the antibody is selective for the VMAT-2 variant polypeptide set forth in SEQ ID NO: 4.

### Microarrays

The presence of a VMAT-2 disease variant can optionally be detected using arrays including DNA microarrays and tissue microarrays. A “microarray” as used herein refers to an ordered set of probes fixed to a solid surface that permits analysis such as gene analysis of a plurality of genes. A DNA microarray refers to an ordered set of DNA fragments fixed to the solid surface. For example, in one embodiment the microarray is a gene chip. In another embodiment the microarray is a microarray comprising single nucleotide polymorphisms, also known as a SNP microarray.

### Kits

Another aspect of the disclosure is a kit for screening for, diagnosing the presence of, or detecting a risk of developing, a VMAT-2 deficiency disease. In one embodiment the kits comprise, one or more isolated nucleic acid molecules and/or compositions described herein and instructions for use.

In an embodiment the kit comprises an isolated nucleic acid molecule or composition that specifically hybridizes to a VMAT-2 disease associated variant, e.g. a probe. In an embodiment the nucleic acid molecule comprises SEQ ID NO:2 or a portion thereof or the complement thereof. In another embodiment, the nucleic acid molecule comprises a detectable label such as a fluorescent molecule. In a further embodiment, the kit comprises an isolated nucleic acid molecule useful as a primer. In an embodiment, the primer is selected from all or part of (e.g. at least 10 or 15 nucleotides, or any number in between) of SEQ ID NO: 1, 2, 5 and 6. In another embodiment the kit comprises at least two nucleic acids wherein one hybridizes to a wildtype or non-disease associated VMAT-2 containing molecule and the other hybridizes to a VMAT-2 disease associated variant containing molecule. In another embodiment the at least two isolated nucleic acids are primers for amplifying a sequence comprising a VMAT-2 disease associated variant. In an embodiment, the at least two isolated nucleic acid molecules comprise two or more of all or part of SEQ ID NO: 1, 2, 5 and 6. In a further embodiment, the primers are selected from the group comprising SEQ ID NOs: 5 and 6.

As used herein “all or part of” of a probe or primer refers to the portion sufficient for in the case a probe, sufficient to specifically hybridize to the intended target and in the case of a primer, sufficient to prime amplification of the intended template.

In other embodiments the kit comprises a binding agent such as an antibody that specifically binds a VMAT-2 disease associated variant polypeptide and instructions for use. In a further embodiment the kit comprises an isolated antibody specific for an epitope present in a VMAT-2 disease associated variant that is not present in a non-disease associated or wild-type VMAT-2.

In certain embodiments, the kit is a diagnostic kit for medical use. In other embodiments, the kit is a diagnostic kit for laboratory use.

In another aspect the disclosure provides a commercial package comprising an isolated nucleic acid or composition described herein and instructions for use.

### Assay for Identifying Additional VMAT-2 Disease Associated Variants

Other VMAT-2 disease associated variants are identified by screening other populations for VMAT-2 mutations that are infrequently or not present in non-diseased subjects, e.g. normal population without VMAT-2 associated disease. Sequence comparison between subjects known to have VMAT-2 associated disease and subjects known not have VMAT-2 associated disease readily identify additional mutations.

Accordingly, in an embodiment, the disclosure provides a method for identifying VMAT-2 disease associated variants comprising determining whether there is a germline alteration in the sequence of VMAT-2 gene or a VMAT-2 gene regulatory sequence in a sample of a subject, wherein the subject has or is suspected of having a VMAT-2 associated disease. In an embodiment, the sequences of the VMAT-2 gene or VMAT-2 gene regulatory sequence in the sample is compared with the sequence of one or more wild type VMAT-2 gene sequences. In another embodiment, determining the germline mutation comprises determining the sequence of a VMAT-2 gene transcript. In another embodiment, the sequence of the VMAT-2 gene transcript is compared with the sequence of one or more wild type VMAT-2 gene transcript sequences and/or a transcript described herein. In an embodiment, the disclosure provides a method for identifying VMAT-2 disease associated variants comprising amplifying a VMAT-2, gene or transcript or part thereof from a sample of a subject, comparing the amplified region to a control population, wherein a mutation that is detected in the sample and is rare or undetected in the control population is a VMAT-2 disease associated variant. In an embodiment, one or more of the nucleic acids of SEQ ID NOs: 5 and 6 are used to amplify a VMAT-2 gene or transcript or part thereof. In another embodiment, the part thereof comprises or corresponds to an exon and/or exon/intron boundary. In another embodiment, the part thereof comprises intronic VMAT-2 sequence.

The VMAT-2 mutation is optionally a deletion mutation, a missense mutation, a point mutation, and/or a mutation that affects VMAT-2 expression levels.

In an embodiment, the method for identifying VMAT-2 disease associated variants comprises detecting the level and/or sequence of an expression product of VMAT-2 in the sample.

In another embodiment, the disclosure provides a method for identifying a VMAT-2 disease associated variant comprising determining whether there is an amino acid alteration in the VMAT-2 polypeptide in the sample compared to the sequence of wild type VMAT-2 polypeptide.

### Screening Assay for Identifying Substances Useful for Treating a VMAT-2 Deficiency Disease

The application also includes screening assays for detecting substances that target or bind VMAT-2. The application also includes screening assays for detecting substances that target or bind a VMAT-2 disease associated variant, which are useful to treat a VMAT-2 deficiency disease. These assays may be in in vitro or in vivo format. In a suitable embodiment the application provides a cell based assay for evaluating whether a candidate compound is capable of binding a VMAT-2 disease associated variant.

Accordingly, the application provides a method of identifying substances which bind a VMAT-2 disease associated variant comprising:

a) contacting a VMAT-2 disease associated variant with a test substance, under conditions which allow for formation of a complex between the VMAT-2 disease associated variant and the test substance, and

b) detecting for complexes between VMAT-2 disease associated variant and the test substance, wherein the presence of complexes indicates that the test substance binds the VMAT-2 disease associated variant.

### Treatment

The inventors have found that treating subjects with a VMAT-2 deficiency disease with a dopamine agonist lessens the associated symptoms.

Accordingly, the disclosure provides a method for treatment for subjects with a VMAT-2 deficiency disease and/or a risk of developing a VMAT-2 deficiency disease comprising administering an effective amount of a dopamine agonist to the subject.

The disclosure also provides a method for treatment for subjects with a VMAT-2 deficiency disease and/or a risk of developing a VMAT-2 deficiency disease comprising:
a) detecting a VMAT-2 disease associated variant in a subject according to a method described herein;
b) administering an effective amount of a dopamine agonist to the subject having the VMAT-2 disease associated variant.

In a further embodiment, the disclosure provides use of a dopamine agonist for a subject comprising a VMAT-2 disease associated variant. Optionally, the VMAT-2 disease associated variant is detected according to a method described herein.

In one embodiment, the dopamine agonist is a dopamine receptor agonist, namely a compound that activates dopamine receptors in the absence of dopamine. Examples of dopamine agonists include, but are not limited to, apomorphine, pramipexole and ropinirole. In a further embodiment, the dopamine agonist is pramipexole.

The disclosure further provides a method for treatment for subjects with a VMAT-2 deficiency disease and/or a risk of developing a VMAT-2 deficiency disease comprising:
a) detecting a VMAT-2 disease associated variant in a subject according to a method described herein;
b) administering an effective amount of a compound to the subject having the VMAT-2 disease associated variant, wherein the compound treats at least one symptom associated with the VMAT-2 deficiency disease.

In one embodiment, the symptom associated with the VMAT-2 deficiency disease is depression and the compound is an antidepressant including, but not limited to, Selective serotonin reuptake inhibitors (SSRIs), Serotonin-norepinephrine reuptake inhibitors (SNRIs), Serotonin antagonist and reuptake inhibitors (SARIs), Norepinephrine reuptake inhibitors (NRIs), Norepinephrine-dopamine reuptake inhibitors (NDRIs), Norepinephrine-dopamine releasing agents (NDRAs), Tricyclic antidepressants (TCAs), Tetracyclic antidepressants (TeCAs) and Monoamine oxidase inhibitors (MAOIs).

The term “effective amount” as used herein means an amount sufficient to achieve the desired result and accordingly will depend on the ingredient and its desired result. Nonetheless, once the desired effect is known, determining the effective amount is within the skill of a person skilled in the art. For example, as used herein an “effective amount of the dopamine agonist” is optionally the amount of dopamine agonist that is sufficient to treat a subject who suffers from a VMAT-2 associated disease.

The terms “treating” or “treatment” as used herein, and as are well understood in the art, mean an approach for obtaining beneficial or desired results, including clinical results. Beneficial or desired clinical results can include, but are not limited to, alleviation or amelioration of one or more symptoms or conditions, diminishment of extent disease, stabilizing (i.e. not worsening) the state of disease, delaying or slowing disease progression, amelioration or palliation of the disease state, diminishment of the reoccurrence of disease, and remission (whether partial or total), whether detectable or undetectable. Treatment methods optionally comprise administering to a subject a therapeutically effective amount of a compound or active agent (for example, dopamine agonists such as pramipexole) and optionally consists of a single administration, or alternatively comprise a series of applications. The length of the treatment period depends on a variety of factors, such as the severity of the condition, the age of the patient, the concentration of the compound, the activity of the compound, and/or a combination thereof. It will also be appreciated that the effective dosage of the compound used for the treatment or prophylaxis may increase or decrease over the course of a particular treatment or prophylaxis regime. Changes in dosage may result and become apparent by standard diagnostic assays known in the art. In some instances, chronic administration may be required. For example, the compound may be administered to the subject in an amount and for a duration sufficient to treat the patient.

A dopamine agonist may be administered to a subject with a VMAT-2 deficiency disease according to standard dosages and treatment regimes as known in the art. In one embodiment, pramipexole is administered to a subject in need thereof at a daily dosage of 0.125 mg to 6.0 mg per day. Pramipexole is optionally administered once, twice or three times a day. For example, pramipexole is optionally administered in an amount of 1.5 to 4.5 mg/day administered in equally divided doses three times per day.

The following non-limiting examples are illustrative of the present disclosure:

### EXAMPLES

### Example 1

### Case Report

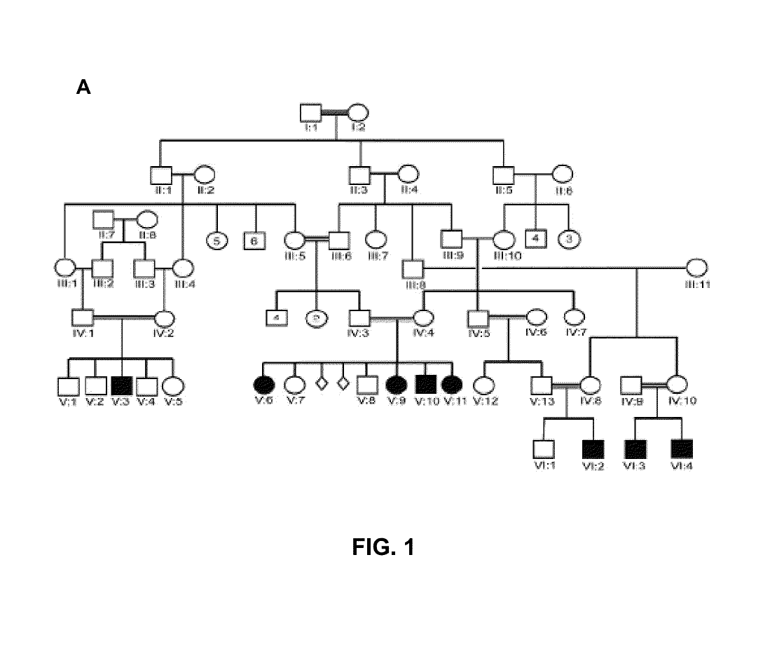

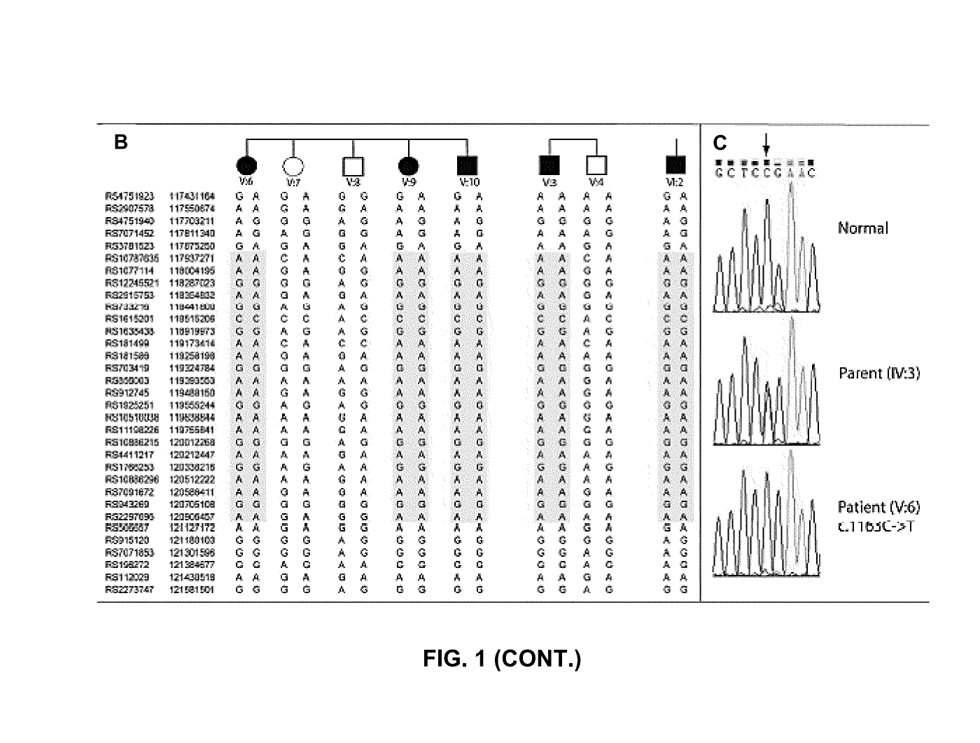

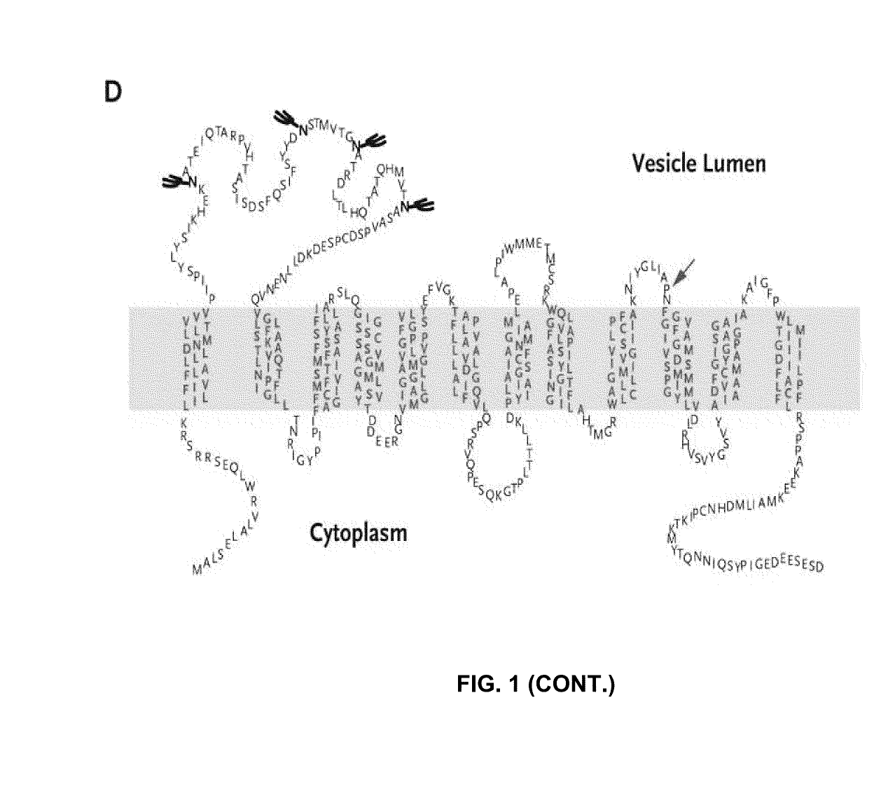

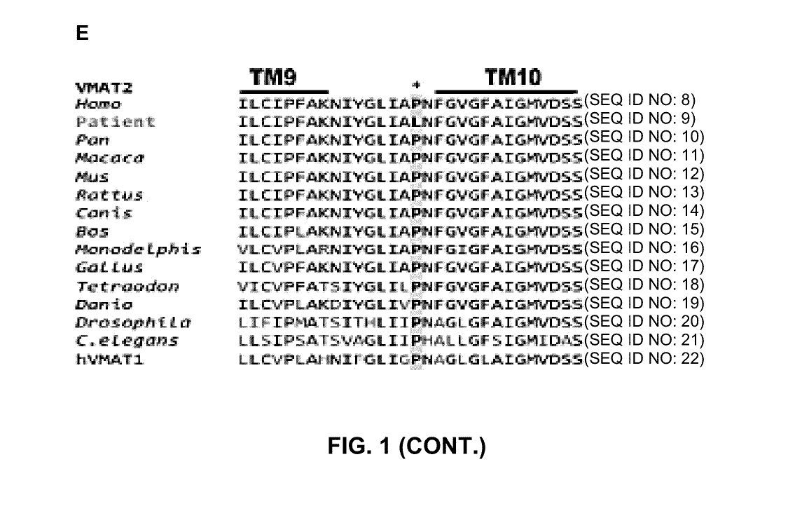

Eight children of an extended consanguineous Saudi family shared identical clinical symptoms of a complex movement disorder inherited in autosomal recessive fashion (FIG. 1A and Table 2). The parents were unaffected but at least five suffer from clinical depression.

The index case was a 16-year-old female with global developmental delay and abnormal movements. She had first been brought to medical attention at four months of age with hypotonia, loss of acquired head control, and paroxysmal stereotyped episodes of persistent eye deviation and crying lasting hours. Video-EEG monitoring had excluded seizures, and a symptom diagnosis of oculogyric crisis had been made. Development had been normal initially, but had slowed after presentation. The girl sat at 30 months, crawled at four years, and walked at 13 years. At 16, she was experiencing fatigue, excessive diaphoresis, profuse nasal and oropharyngeal secretions, noisy breathing, hypernasal speech, poor distal perfusion, cold extremities, disrupted sleep, hypotonia, dysarthria, and ataxia. There was no diurnal variation and no improvement with vitamin B6 or folinic acid. Neurological examination revealed ptosis, hypomimia, facial dyskinesia, and limited upward gaze. She had axial hypotonia and appendicular hypertonia specifically involving extensor muscles of upper and lower extremities. Deep tendon reflexes were 2+/4 and plantars flexor. Coordination testing revealed a fine tremor, and dysdiadochokinesia in upper and lower extremities. Gait was parkinsonian with typical shuffling, posture was stooped, and postural reflexes diminished. She walked with bilateral alternating dystonia of hands and feet with intermittent toe walking and foot inversion, and was unable to tandem walk.

Basic blood tests, metabolic screens (Table 3), repeat video-EEG, MRI, and MR spectroscopy were normal. Lumbar puncture in a two-year-old affected sibling showed normal neurotransmitter metabolites, intermediates, and precursors (Table 4). Urine neurotransmitter profile, however, revealed elevated levels of monoamine metabolites [5-HIAA=17.6 μg/dL (reference range: 0-6 μg/dL); HVA=14.1 μg/mg Cr (0-13.4 μg/mg Cr)] and decreased measurable monoamines [norepinephrine=1.1 μg/dL (4-29 μg/dL); dopamine=19 μg/dL (40-260 μg/dL)) (Table 4).

On the basis of the parkinsonism and the diminished urine dopamine, the proband and three younger affected siblings were treated with L-DOPA/carbidopa, which within one week resulted in major deterioration, with the appearance of intense chorea and worsened dystonia. Discontinuation of the medication led to rapid return to baseline in all four children.

### Methods

### Genetic Studies

More than 300,000 single nucleotide polymorphisms (SNPs) were genotyped in eight family members, V:2,3,6,7,8,9,VI:2 (FIG. 1A) (using a 300K Illumina SNP microarray), followed by homozygosity mapping to identify the homozygous loci shared by the affected children. A subset of 2500 SNPs with HapMap minimal allele frequency >0.4 and average spacing of ˜1.0 Mb was selected for parametric linkage analysis. Sanger sequencing of candidate gene exons was performed to identify the mutation, whole-exome sequencing (Agilent V4 50 Mb capture kit, Illumina Hiseq 2000 sequencing) to exclude other mutations, and Taqman genotyping to confirm absence of the mutation in controls.

### Functional Analysis of P387L Mutant Vmat-2

A construct was engineered encoding VMAT-2 containing the P387L substitution, and carried out an assay of vesicular serotonin uptake in a heterologous cell system. Transport mediated by VMAT-2 was measured by incubating membrane preparations with tritiated serotonin, followed by rapid washing and filtration to retain vesicles with trapped substrate.

### Vector Construction

Human VMAT2 cDNA sequence was acquired from the Mammalian

Gene Collection and subcloned into pcDNA3.1A (Invitrogen). The P387L (c.1160C->T) mutation was introduced by site-directed mutagenesis using the QuikChange Site-Directed Mutagenesis kit (Strategene) and confirmed by sequencing.

### Isolation of Lysate Containing Microsomes

Cos 7 cells were transfected at 80% confluency using Fugene HD (Roche). After 48 h, cells were washed in 1×PBS and pelleted. Cell pellets were immediately resuspended in 320 mM sucrose-HEPES buffer (pH 7.4) and sonicated by 20×1 s pulses. Lysates were centrifuged at 4,000 g for 5 min, and supernatants were stored at −80° C. until use.

### Tritiated Serotonin Uptake Assay

Reaction buffer contained 150 mM choline gluconate, 10 mM HEPES-Tris (pH 7.4), 2 mM Mg-ATP, and 90 nM 3H-serotonin (New England Biolabs). Where indicated, reaction buffer also contains 10 μM reserpine. To each tube was added 10 μL of microsomal lysate, followed by incubation at 30° C. for the specified time. Reactions were stopped by rapid filtration, and retained serotonin measured by scintillation counting.

### Results

### Mutation Identification

Homozygosity mapping identified a single homozygous 3.2-Mb interval in 10q25.3-26.11 shared by five affected family members but not by unaffected members (FIG. 1B). Parametric linkage analysis produced a significant lod score of 4.1 in this region. Another locus, on chromosome 3, yielded a significant LOD score of 3.1 but did not correspond to a region of shared homozygosity. Exons and exon-intron boundaries of eight genes known to have neuronal functions were sequenced and observed a novel variant (c.1160C→T) in exon 13 (FIG. 1C) was observed, predicted to result in a substitution of proline with leucine at position 387 (p.P387L), in VMAT-2. The variant is homozygous in affected individuals and not in 78 unaffected members of the family, 26 of whom carry the variant in heterozygous state. Whole-exome sequencing in the proband was also performed, which independently identified the SLC18A2 change and revealed no other novel non-synonymous variant in the linked region of shared homozygosity. SLC18A2 c.1160C→T is not present in datasets of sequenced genomes including the 1000-genome database. In addition, as one of the most studied candidate genes for involvement in Parkinson's disease, SLC18A2 was previously screened in 704 healthy individuals of diverse ethnic backgrounds and 452 Parkinson's disease patients¹¹⁻¹³, none of whom had the c.1160C→T change. Collectively, these results show that SLC18A2 c.1160C→T is the causative defect in this family.

### Functional Characterization of P387L Mutant Vmat-2

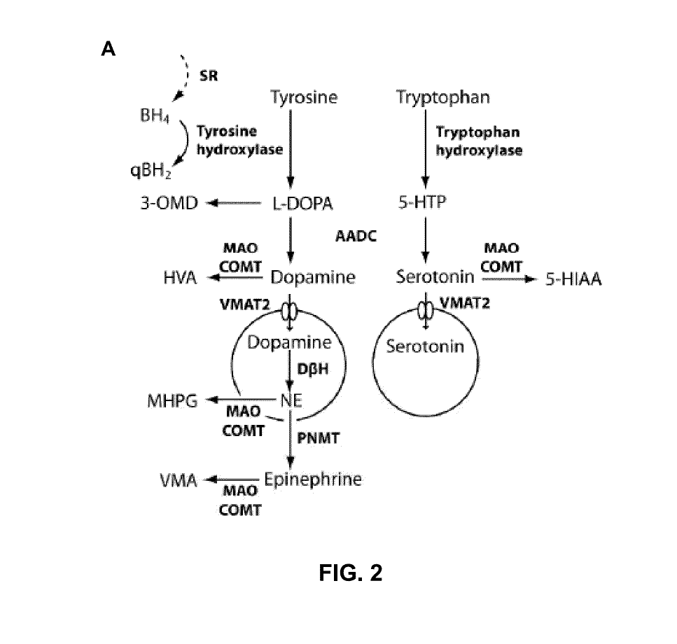

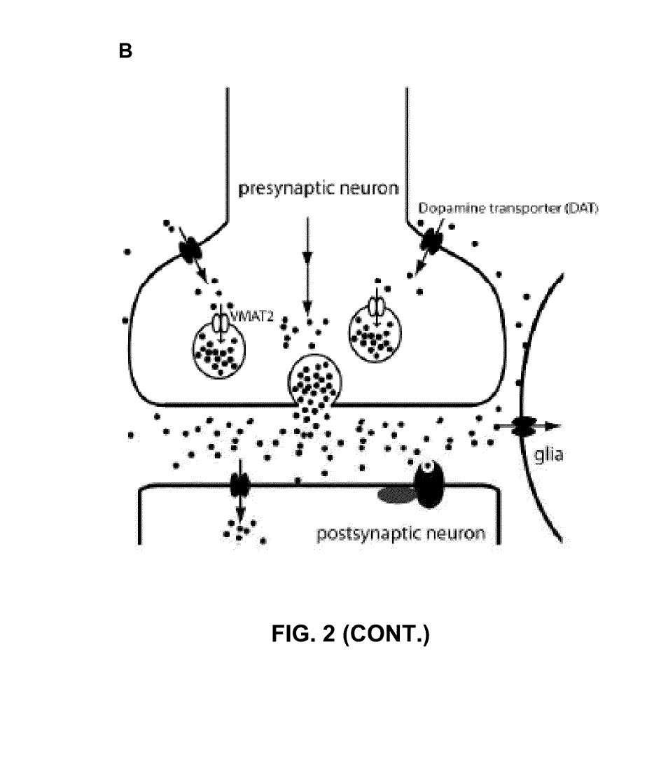

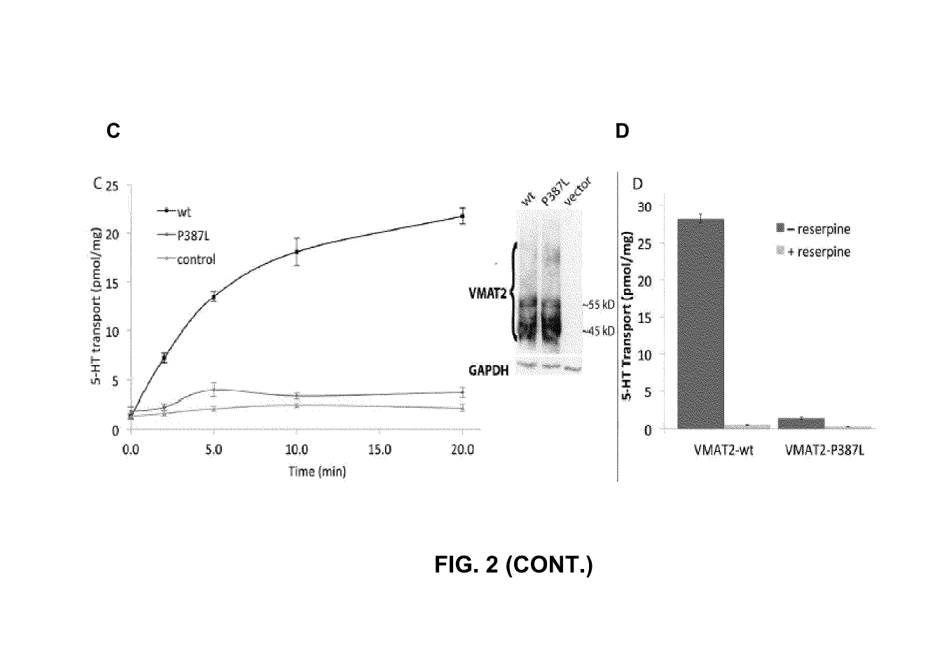

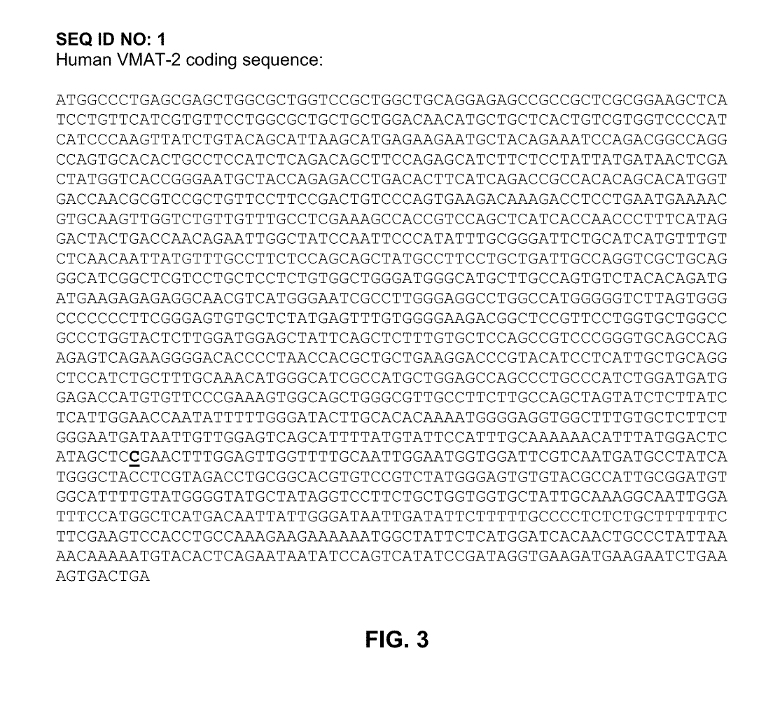

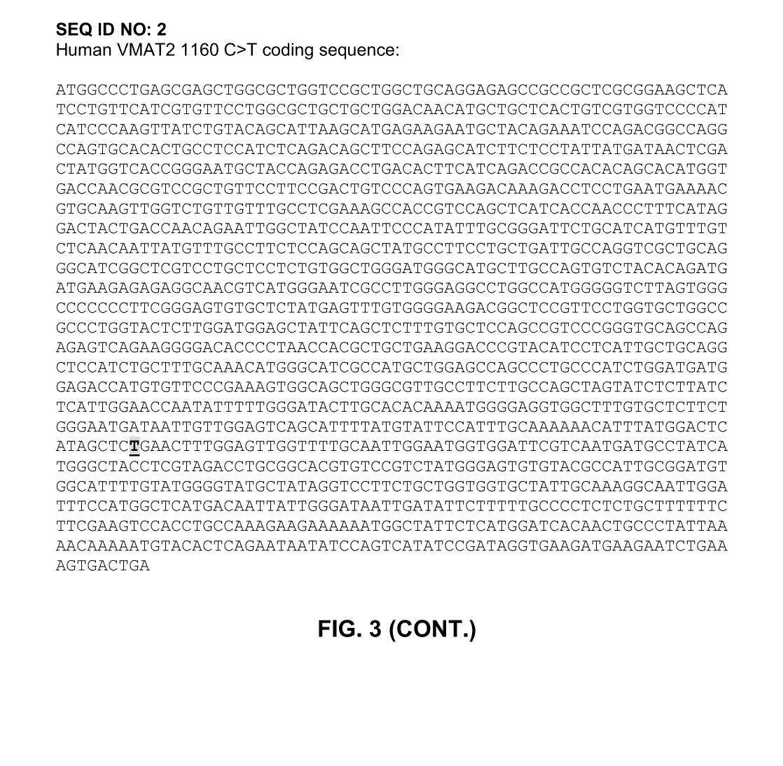

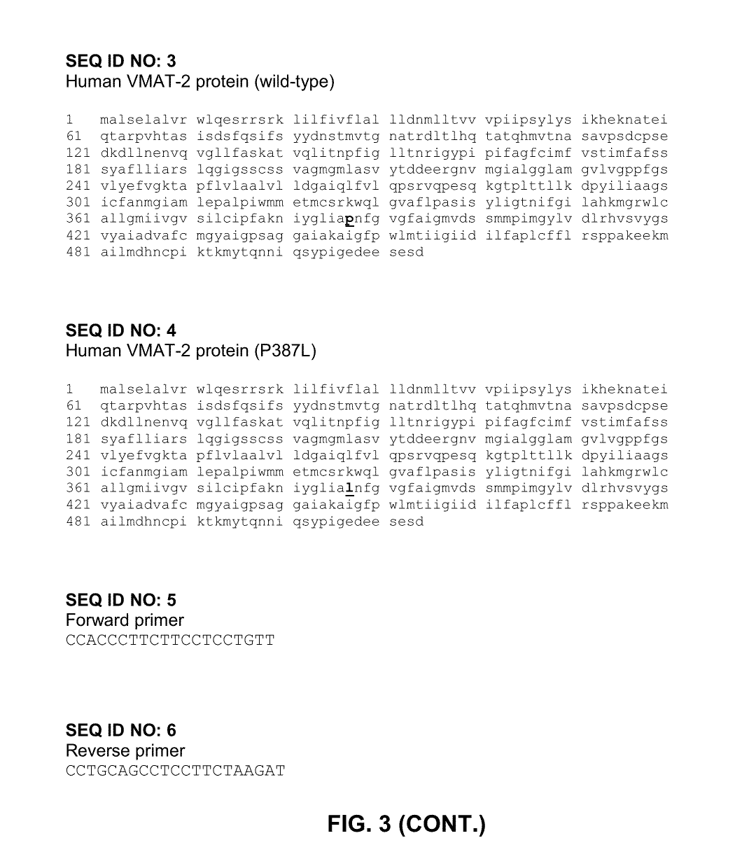

SLC18A2 encodes the vesicular monoamine transporter-2 (VMAT-2) protein located in membranes of monoamine synaptic vesicles (FIGS. 1D, 2A and 2B). Proline residues adjacent to transmembrane segments have major structural effects and are overrepresented among residues subject to disease-causing substitutions.¹⁴ Pro387 is immediately adjacent a transmembrane segment (FIG. 1D). Sequence alignment shows that Pro387 is highly conserved through evolution and thus suggest that its substitution is likely to be deleterious. It is also conserved in the paralogous protein VMAT-1 and in CAT-1 of C. elegans CAT-1—the single vesicular monoamine transporter in nematodes (FIG. 1E).¹⁵ Interestingly, the residue is not conserved in the vesicular acetylcholine transporter (VAChT), which maintains 39% identity to VMAT2, showing that Pro387 has a specific role in monoamine transport.

To determine the effect of the P387L mutation on VMAT-2 transport activity, we transiently and separately expressed non-mutant and mutant human VMAT2 in Cos cells. Immunoblotting membrane preparations confirmed equivalent levels of mature glycosylated VMAT2 in parallel transfections, suggesting no major defect in protein processing. However, P387L-VMAT-2 showed dramatically decreased activity compared with non-mutant VMAT-2

(FIG. 2C). Use of the specific VMAT inhibitor reserpine confirmed that P387L-VMAT-2 still exhibits some weakly measurable uptake (FIG. 2D). Thus, the P387L mutation results in severe, but not complete, loss-of-function.

### Treatment

Defective monoamine loading into synaptic vesicles, and therefore neurotransmission, was consistent with symptoms of monoamine deficiency in affected members of the family, despite their normal levels of brain monoamine. With this insight treated the proband was treated with a direct dopamine receptor agonist (pramipexole), which resulted, within one week, in dramatic and sustained disappearance of parkinsonism and dystonic attacks and improvement of other symptoms (Table 1). The younger siblings were then treated, who also showed improvement. It seemed that, the younger the affected child, the more substantial the recovery (Table 1). The affected children are now at their 32^{nd} month of treatment with continuing benefit.

TABLE 1
The effect of age at dopamine agonist initiation on disease course

| | Age | | | |
|---|---|---|---|---|
| Symptom | 18 y | 11 y | 7 y | 3 y |
| Cognition | Mildly | Mildly | Moderately | Greatly improved. |
| and ability | improved | improved | improved | Able to make |
| to learn | | | | stories from pictures |
| Occulogyric | No further | No further | No further | No further |
| crises | events, requires | events | events | events |
| | dose/weight | | | |
| | higher than her | | | |
| | siblings | | | |
| Dystonia | Gait dystonia | Gait | Gait dystonia | Gait |
| | persists | dystonia | persists | dystonia |
| | | persists | | improved |
| Parkinsonism | Improved | Improved | Improved | Improved |
| Fine motor | Improved | Improved | Learning to hold | Able to write, |
| skills | coordination, | coordination, | a pen and drink | learning to |
| | able to feed self, | learning | from a cup | read |
| | drink from cup, | to hold a | independently. | |
| | and hold a pen. | pen. Unable | Unable to write | |
| | Improved | to write or | or read | |
| | handwriting | read | | |
| Language | Dysarthric | No | Mama and Papa | Normal language |
| and speech | | language | | development and |
| | | development | | mild dysarthria |
| Gait | Improved | Started | Started walking | Started walking |
| | posture and | walking | within days of | within |
| | fatigue (had | within days | treatment | days of |
| | started walking at | of | | treatment |
| | age 13) | treatment | | |

TABLE 2
Cardinal symptoms and signs of patients
with VMAT2 mutation in this study

Onset at 4 months of age.
Hypotonia.
Hypomemia
Paucity of movement.
Oculogyric crises.
Attacks of Dystonia.
Dysarthria.
Ataxia and incoordination.
Excessive diaphoresis.
Profuse nasal and oropharyngeal secretions.
Poor distal perfusion and cold extremities.
Disrupted sleep.
Mild cognitive impairment.
No diurnal variation.
Evolution of the movement disorder by age 11 years into a picture closely
resembling Parkinson's disease, with dyskinesia.

TABLE 3
Serum metabolic screen performed; results all normal

Amino acids
Vitamin B12
Biotinidase
Copper
Ceruloplasmin,
Lead,
Very long chain fatty acids
Carnitine
Ammonia
Lactate
Pyruvate.

TABLE 4
Tested CSF and urine neurotransmitters or their metabolites.

| CSF - | | | HVA/5-HIAA | | | |
|---|---|---|---|---|---|---|
| Normal | 5-HIAA | HVA | ratio | 3-OMD | 5-OHTrp | 5-HT |
| range | 74-345 nM | 233-928 nM | 1.5-4.1 | <150 nM | <25 nM | no reference |
| CSF - | 169 nM | 314 nM | 1.9 | 20 nM | 12 nM | <5 nM |
| Patient | | | | | | |

| Urine - | | | | VMA | HVA | |
|---|---|---|---|---|---|---|
| Normal | 5-HIAA | Norepinephrine | Epinephrine | 0-12.9 μg/mg | 0-13.4 μg/mg | Dopamine |
| range | 0-6 μg/dl | 4-29 μg/dl | 0.0-6.0 μg/dl | Cr | Cr | 40-260 μg/dl |
| Urine - | 17.6 μg/dl | 1.1 μg/dl | 0.5 μg/dl | 5.5 μg/mg Cr | 14.1 μg/mg Cr | 19 μg/dl |
| Patient | | | | | | |

HIAA, hydroxyindoleacetic acid;
HVA, homovanilic acid;
OMD, O-methyldopa;
OHTrp, hydroxytryptophan;
HT, hydroxytyramine;
Cr, creatinine

### Discussion

The mutation in SLC18A2 described here is expected to affect monoamine neurotransmission and thus result in a phenotype that has overlap with all monoamine disorders. Because movement disorder is conspicuous among symptoms of monoamine disturbance, the clinical picture of the disease that is described here is closest to diseases affecting dopamine—chiefly dopamine transporter (DAT), tetrahydrobiopterin (BH4), tyrosine hydroxylase (TH), and aromatic amino acid decarboxylase (AADC) deficiencies (FIGS. 2A and 2B). The phenotype of the affected siblings has particular similarity to AADC deficiency in that it improves with direct dopamine agonism but not with L-DOPA, although the siblings showed a greater improvement than that typically observed in those with AADC deficiency treated with dopamine, and rather than showing a lack of response to L-DOPA, the siblings exhibited a worsening of symptoms. Two other features that distinguish the disease described here from AADC deficiency are the absence of improvement with the AADC enzyme cofactor vitamin B6, and absence of worsening in the evening, which in AADC deficiency is the result of neurotransmitter depletion due to insufficient production.^{1,4,7,8}

The accepted standard diagnostic test in patients with suspected diseases of monoamine metabolism is the measurement of monoamine metabolites in the CSF. Because each specific defect results in a particular metabolite profile, this single test specifies the disease.^{2,3,6} Analysis of monoamines or their metabolites in urine is not reliable in the diagnosis of monoamine neurotransmitter diseases^{2,3,6}, except in one—AADC deficiency—where increased 3-OMD with decreased VMA (see FIG. 2A) in the proper clinical context is highly suggestive and generally confirmed by mutation analysis.^{1,4,7,8} In the present condition, the urine shows abnormalities because VMAT2 functions also outside the central nervous system, including in the peripheral nervous system, adrenal medulla, and platelets.¹⁶ The detection of abnormalities in the urine and not the CSF may reflect differences in monoamine and metabolite stabilities, processing, and normal value ranges between brain and periphery. In any case, it appears that the metabolically and clinically close diseases, AADC and VMAT2 deficiencies, could be screened for by urine testing, and then confirmed by gene sequencing, thus obviating the need for a lumbar punch.

Direct characterization of the mutant VMAT2 protein in this study revealed a severe detriment of vesicular transporter function, which could be due to poor incorporation of the transporter into vesicle membranes, or loss of activity. Proline-to-leucine substitutions are generally considered to be deleterious to organismal fitness (PAM250=−3; BLOSUM62=−3)^{17,18} based on analyses of amino-acid substitutions in evolutionarily conserved proteins and to be damaging to protein function owing to physicochemical difference (Grantham value=98).¹⁹ Proline places unique constraints on peptide backbone flexibility particularly with respect to insertions of adjacent transmembrane segments.¹⁴

A complete knockout Vmat2 in mice results in absent exocytotic monoamine neurotransmission; the mutant animals feed poorly and die within days after birth.^{20,21} By contrast, mice that express just 5% native Vmat2 levels live to adulthood and develop minor age-related motor deficits over time.²² The phenotypic spectrum of VMAT2 deficiency in mice is therefore broad, and consistent with large decreases in protein function being required to cause severe motor symptoms.

The motor phenotype is correctable, and without being bound by theory, suggests that this correction is disease-stage dependent. If true, this dependency could be due to irreversibly perturbed reorganization of dopamine pathways in brains subjected to chronic monoamine neurotransmission deficiencies during active brain development. While the improvement in the patients in this study is striking, it is not complete, likely because of monamine deficiency during development, and also to ongoing deficiencies of the non-dopamine amines and of regulated dopamine release.

Heterozygous mice possessing a single Slc18a2 allele exhibit no motor phenotype, but express a depressive behavioral phenotype.²³ A very high rate of depression was found in the parents of the patients (five of five parents interviewed). This is seen also in parents of patients with AADC deficiency and thought to be caused by clinically significant reductions in serotonin in these individuals with hemizygous defects in the serotonin pathway^{1,4,7,8.}

The initial selection of treatment of the affected children on the basis of clinical phenotype alone (parkinsonism) led to severe, immediate worsening of the movement disorder. This was likely caused by the known toxicity of elevated amounts of dopamine, in particular to dopaminergic neurons.²⁴ Subsequent identification of the underlying pathophysiology allowed the rational selection of an appropriate treatment. A related severe disorder, sepiapterin reductase deficiency (see FIG. 2A) was recently diagnosed by whole genome sequencing a pair of siblings who had remained long-undiagnosed and therefore untreated because of the difficulties in obtaining a precise diagnosis for rare diseases. Diagnosis allowed treatment and recovery of these children.²⁵

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES

1. Brun L, Ngu L H, Keng W T, et al. Clinical and biochemical features of aromatic L-amino acid decarboxylase deficiency. Neurology 2010; 75:64-71.
2. Hyland K. Clinical utility of monoamine neurotransmitter metabolite analysis in cerebrospinal fluid. Clinical chemistry 2008; 54:633-41.
3. Kurian M A, Gissen P, Smith M, Heales S, Jr., Clayton P T. The monoamine neurotransmitter disorders: an expanding range of neurological syndromes. Lancet Neurol 2011; 10:721-33.
4. Lee H C, Lai C K, Yau K C, et al. Non-invasive urinary screening for aromatic L-amino acid decarboxylase deficiency in high-prevalence areas: a pilot study. Clin Chim Acta 2012; 413:126-30.
5. Longo N. Disorders of biopterin metabolism. J Inherit Metab Dis 2009; 32:333-42.
6. Pons R. The phenotypic spectrum of paediatric neurotransmitter diseases and infantile parkinsonism. J Inherit Metab Dis 2009; 32:321-32.
7. Pons R, Ford B, Chiriboga C A, et al. Aromatic L-amino acid decarboxylase deficiency: clinical features, treatment, and prognosis. Neurology 2004; 62:1058-65.
8. Swoboda K J, Hyland K, Goldstein D S, et al. Clinical and therapeutic observations in aromatic L-amino acid decarboxylase deficiency. Neurology 1999; 53:1205-11.
9. Yeung W L, Wong V C, Chan K Y, et al. Expanding phenotype and clinical analysis of tyrosine hydroxylase deficiency. Journal of child neurology 2011; 26:179-87.
10. Kurian M A, Zhen J, Cheng S Y, et al. Homozygous loss-of-function mutations in the gene encoding the dopamine transporter are associated with infantile parkinsonism-dystonia. J Clin Invest 2009; 119:1595-603.
11. Burman J, Tran C H, Glatt C, Freimer N B, Edwards R H. The effect of rare human sequence variants on the function of vesicular monoamine transporter 2. Pharmacogenetics 2004; 14:587-94.
12. Glatt C E, DeYoung J A, Delgado S, et al. Screening a large reference sample to identify very low frequency sequence variants: comparisons between two genes. Nature genetics 2001; 27:435-8.
13. Iwasa H, Kurabayashi M, Nagai R, Nakamura Y, Tanaka T. Multiple single-nucleotide polymorphisms (SNPs) in the Japanese population in six candidate genes for long QT syndrome. J Hum Genet. 2001; 46:158-62.
14. Partridge A W, Therien A G, Deber C M. Missense mutations in transmembrane domains of proteins: phenotypic propensity of polar residues for human disease. Proteins 2004; 54:648-56.
15. Duerr J S, Frisby D L, Gaskin J, et al. The cat-1 gene of Caenorhabditis elegans encodes a vesicular monoamine transporter required for specific monoamine-dependent behaviors. J Neurosci 1999; 19:72-84.
16. Eiden L E, Weihe E. VMAT2: a dynamic regulator of brain monoaminergic neuronal function interacting with drugs of abuse. Ann N Y Acad Sci 2011; 1216:86-98.
17. Dayhoff M O, Schwartz R M, Orcutt B C. A model of evolutionary change in proteins. In: Dayhoff M O, ed. Atlas of Protein Sequence and Structure. Washington: National Biomedical Research Foundation; 1978:345-52.
18. Henikoff S, Henikoff J G. Amino acid substitution matrices from protein blocks. Proc Natl Acad Sci USA 1992; 89:10915-9.
19. Grantham R. Amino acid difference formula to help explain protein evolution. Science 1974; 185:862-4.
20. Wang Y M, Gainetdinov R R, Fumagalli F, et al. Knockout of the vesicular monoamine transporter 2 gene results in neonatal death and supersensitivity to cocaine and amphetamine. Neuron 1997; 19:1285-96.
21. Fon E A, Pothos E N, Sun B C, Killeen N, Sulzer D, Edwards R H. Vesicular transport regulates monoamine storage and release but is not essential for amphetamine action. Neuron 1997; 19:1271-83.
22. Mooslehner K A, Chan P M, Xu W, et al. Mice with very low expression of the vesicular monoamine transporter 2 gene survive into adulthood: potential mouse model for parkinsonism. Molecular and cellular biology 2001; 21:5321-31.
23. Fukui M, Rodriguiz R M, Zhou J, et al. Vmat2 heterozygous mutant mice display a depressive-like phenotype. J Neurosci 2007; 27:10520-9.
24. Jeon S M, Cheon S M, Bae H R, Kim J W, Kim S U. Selective susceptibility of human dopaminergic neural stem cells to dopamine-induced apoptosis. Exp Neurobiol 2010; 19:155-64.
25. Bainbridge M N, Wiszniewski W, Murdock D R, et al. Whole-genome sequencing for optimized patient management. Sci Transl Med 2011; 3:87re3.

1. A method of screening for, diagnosing and/or detecting an increased risk of developing a VMAT-2 deficiency disease in a subject comprising detecting the presence of a VMAT-2 disease associated variant in a sample from the subject, wherein the presence of a VMAT-2 disease variant is indicative that the subject has a VMAT-2 deficiency disease and/or an increased risk of developing a VMAT-2 deficiency disease.

2. The method of claim 1, wherein detecting the presence of a VMAT-2 disease associated variant comprises assaying the sample for the presence of and detecting a variant in a VMAT-2 nucleic acid molecule.

3. The method of claim 2, wherein assaying the sample comprises hybridizing a probe and/or primer to the VMAT-2 nucleic acid molecule

4. The method of claim 1 wherein the VMAT-2 disease associated variant comprises a mutation of a nucleotide corresponding to residue 1160 of SEQ ID NO: 1.

5. The method of claim 4, wherein the mutation comprises mutation of cysteine to thymidine.

6. The method of claim 1, wherein the VMAT-2 disease associated variant comprises a mutation of an amino acid in a VMAT-2 polypeptide.

7. The method of claim 6, wherein the amino acid corresponds to position

8. The method of claim 7, wherein the mutation is P387L.

9. The method of claim 1, wherein the VMAT-2 disease associated variant is detected by one or more of: genotyping, using a probe that hybridizes to a VMAT-2 disease associated variant nucleic acid, PCR, RT-PCR, NASBA, a binding agent, and/or microarray.

10. The method of claim 1, wherein the subject is presymptomatic, has one or more clinical symptoms or clinical features associated with a VMAT-2 deficiency disease and/or has been diagnosed with a VMAT-2 deficiency disease.

11. The method of claim 1 further comprising treating a subject with a VMAT-2 deficiency disease by administering an effective amount of a dopamine agonist to the subject.

12. The method of claim 11, wherein the dopamine agonist is pramipexole.

13. An isolated nucleic acid, wherein the nucleic acid hybridizes to:
a. a RNA product of a VMAT-2 variant associated with a VMAT-2 deficiency disease
b. a nucleic acid sequence complementary to a); and/or
c. a nucleic acid sequence corresponding to a).

14. The isolated nucleic acid of claim 13, wherein the nucleic acid comprises all or part of the nucleic acid sequence set forth in SEQ ID NO: 1 and has a thymidine at a position corresponding to residue 1160 of SEQ ID NO:1.

15. The isolated nucleic acid of claim 13, wherein the isolated nucleic acid is a primer or a probe and the isolated nucleic acid comprises or consists of a nucleic acid sequence corresponding to at least 5, 10, 15, 20, 30, 40 or 50 contiguous nucleic acid residues of SEQ ID NO: 2, including residue number 1160, or the complement thereof.

16. The isolated nucleic acid of claim 13, comprising:
a) any one of SEQ ID NOs: 1, 2, 5 or 6 and/or combinations or parts thereof, and/or
b) a nucleic acid molecule with at least 80%, 90%, 95%, or 99% sequence identity to a);
wherein the nucleic acid molecule is capable of binding to a VMAT-2 disease associated variant under stringent hybridization conditions.

17. An isolated polypeptide encoded by the isolated nucleic acid of claim 13.

18. A kit for screening for, diagnosing or detecting an increased risk of developing VMAT-2 deficiency disease comprising:
a) a VMAT2 disease variant detection agent; and
b) instructions for use.

19. The kit of claim 18, wherein the detection agent comprises the isolated nucleic acid of claim 13.

20. The kit of claim 18, wherein the detection agent comprises an antibody.
